(19) United States
(12) Patent Application Publication
(10) Pub. No.: US 2005/0209310 A1
(43) Pub. Date: Sep. 22, 2005

(54) Methods for modulating tumor growth and metastasis

(76) Inventors: David J. Chaplin, Watlington (GB); Klaus Edvardsen, Lund (SE); Scott Young, Sudbury, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/020,463

(22) Filed: Dec. 22, 2004

Related U.S. Application Data
(63) Continuation-in-part of application No. 10/027,186, filed on Dec. 20, 2001.
(60) Provisional application No. 60/258,195, filed on Dec. 22, 2000.

(51) Int. Cl. A61K 31/337; A61K 31/28; A61K 31/075

(52) U.S. Cl. 514/449; 514/492; 514/720

(57) ABSTRACT

Methods and pharmaceutical compositions for modulating tumor growth or metastasis and methods for prognosing treatment therewith are provided.

This application is a continuation-in-part of co-pending U.S. patent application Ser. No. 10/027,186, filed Dec. 21, 2001 and entitled “Methods For Modulating Tumor Growth and Metastasis”, which in turn claims priority to U.S. Provisional Application 60/258,195, filed Dec. 22, 2000, entitled “Methods For Modulating Tumor Growth and Metastasis”.

### FIELD OF THE INVENTION

This invention relates to the fields of oncology and improved chemotherapy regimens.

### BACKGROUND OF THE INVENTION

Cellular transformation during the development of cancer involves multiple alterations in the normal pattern of cell growth regulation. Primary events in the process of carcinogenesis involve the activation of oncogene function by some means (e.g., amplification, mutation, chromosomal rearrangement), and in many cases, the removal of anti-oncogene function. In the most malignant and untreatable tumors, normal restraints on cell growth are completely lost as transformed cells escape from their primary sites and metastasize to other locations in the body. One reason for the enhanced growth and invasive properties of some tumors may be the acquisition of increasing numbers of mutations in oncogenes, with cumulative effect (Bear, et al., Proc. Natl. Acad. Sci. USA 86:7495-7499, (1989)).

Alternatively, insofar as oncogenes function through the normal cellular signaling pathways required for organismal growth and cellular function (reviewed in McCormick, et al., Nature, 63:15-16, (1993)), additional alterations in the oncogenic signaling pathways may also contribute to tumor malignancy (Gilks, et al., Mol. Cell Biol. 13:1759-1768, (1993)), even though mutations in the signaling pathways alone may not cause cancer.

Several discrete classes of proteins are known to be involved in bringing about the different types of changes in cell division properties and morphology associated with transformation. These changes can be summarized as, first, the promotion of continuous cell cycling (immortalization); second, the loss of responsiveness to growth inhibitory signals and cell apoptotic signals; and third, the morphological restructuring of cells to enhance invasive properties.

The National Cancer Institute has estimated that in the United States alone, 1 in 3 people will be struck with cancer during their lifetime. Moreover approximately 50% to 60% of people contracting cancer will eventually succumb to the disease. The widespread occurrence of this disease underscores the need for improved anticancer regimens for the treatment of malignancy.

Due to the wide variety of cancers presently observed, numerous anticancer agents have been developed to destroy cancer within the body. These compounds are administered to cancer patients with the objective of destroying or otherwise inhibiting the growth of malignant cells while leaving normal, healthy cells undisturbed. Anticancer agents have been classified based upon their mechanism of action. One type of chemotherapeutic is referred to as a metal coordination complex (e.g. platinum coordination compounds). It is believed this type of chemotherapeutic forms predominantly inter-strand DNA cross-links in the nuclei of cells, thereby preventing cellular replication. As a result, tumor growth is initially repressed, and then reversed. Another type of chemotherapeutic is referred to as an alkylating agent. These compounds function by inserting foreign compositions or molecules into the DNA of dividing cancer cells. As a result of these foreign moieties, the normal functions of cancer cells are disrupted and proliferation is prevented. Another type of chemotherapeutic is an antineoplastic agent. This type of agent prevents, kills, or blocks the growth and spread of cancer cells. Still other types of anticancer agents include mitotic inhibitors, nonsteroidal aromatase inhibitors, bifunctional alkylating agents, etc.

Unfortunately, deleterious side effects are associated with each of these agents. For example, fluorouracil, a commonly used antineoplastic agent causes swelling or redness of normal skin, black or tarry stools, blood in the urine, chest pain, confusion, diarrhea, shortness of breath, and drowsiness. Administration of fluorouracil has also been associated with fever, chills, cough, sore throat, lower back pain, mouth sores, nausea, vomiting, pain and/or difficulty passing urine. Taxanes, mitotic inhibitors which are commonly used for anti-cancer use, have been associated with cardiovascular events such as syncope, rhythm abnormalities, hypertension and venous thrombosis; bone marrow suppression, neutropenia, anemia, peripheral neuropathy arthralgia/myalgia, nausea/vomiting and alopecia, to name only a few.

In addition to their often considerable toxicity, many conventional anticancer agents are ineffective or gradually fail to be effective in treating certain tumors due to the presence of acquired or intrinsic tumor mutations that confer resistance to the chemotherapeutic. Acquired or intrinsic drug resistance is a major complication in cancer chemotherapy and accounts for the failure of chemotherapy to cure the majority of cancer patients (Gottesman et al., Annu Rev. Biochem., 62:385-427 (1993); Van Der Zee, et al., Gynecologic Oncol., 58:165-178 (1995); Casazza et al., Cancer Treat. Res. 87:1-171, (1996)). For example, tumors may acquire resistance to platinum coordination compounds such as cisplatin due to their acquisition of mutations, which cause a decreased intracellular accumulation of cisplatin or increased DNA repair (Chu et al., J. Biol. Chem., 269: 787-790, (1994)). Drug resistance also has significant clinical implications. When cells become resistant to a particular anticancer agent, the doses must be increased, leading to a worsening of drug-associated toxicities.

Combretastatins are another class of anticancer agents. Combretastatins have been isolated from stem wood of the African tree Combretum caffrum (Combretaceae), and are potent inhibitors of microtubulin assembly. Combretastatin A-4 (“CA4”) is significantly active against the US National Cancer Institute's (NCI) murine L1210 and P338 lymphocytic leukemia cell lines. In addition, CA4 was found to compete with combretastatin A-1 (“CA1”), another compound isolated from Combretum caffrum, as a potent inhibitor of colchicine binding to tubulin. CA4 also strongly retards the growth of certain cell lines (ED50 <0.01 (g/ml)) and is a powerful anti-mitotic agent. See U.S. Pat. No. 4,996,237. Since the solubility of the combretastatins is very limited, prodrugs have been developed, such as combretastatin A-4 phosphate and combretastatin A-1 diphosphate (hereinafter “CA4P” and “CA1P” respectively), to increase the solubility, and thus the efficacy of CA-4 and CA-1.

Although CA4P and CA1P have activity as inhibitors of tumor cell proliferation, their primary mechanism of action has been shown to be one of “vascular targeting”, in which the neovasculature of solid tumors is selectively disrupted, resulting in a transient decrease or complete shutdown of tumor blood flow that results in secondary tumor cell death due to hypoxia, acidosis, and/or nutrient deprivation (Dark et al., Cancer Res., 57: 1829-34, (1997); Chaplin et al., Anticancer Res., 19: 189-96, (1999); Hill et al., Anticancer Res., 22(3):1453-8 (2002); Holwell et al., Anticancer Res., 22(2A):707-11, (2002). While effective in killing the vast majority of the tumor mass, some tumors are nonetheless resistant to treatment with CA4P due to a rim of viable tumor tissue which can serve to repopulate the tumor, eventually leading to progression of tumor cell growth (Dark et al., Cancer Res., 57: 1829-34, (1997); Chaplin et al., Anticancer Res., 19: 189-96, (1999)). This rim of surviving tissue is most likely a consequence of the shared normal vessel circulation between the tumor perimeter and neighboring normal tissue. Toxic side effects of CA4P have also been reported.

There is thus a need in the art to provide superior effective anticancer therapies, which minimize patient exposure, combat drug resistance, and reduce the unwanted side effects associated with such agents.

### BRIEF DESCRIPTION OF THE DRAWINGS

FIG. 1: Graph of the antitumor activity of cisplatin and the CA4P disodium salt administered singly in the moderately platinum-resistant M5076DDP murine fibrosarcoma. Tumor was staged to 300 mg at treatment initiation. Cisplatin was administered intravenously (iv), every 4 days for 3 doses (Q4D×3). CA4P was given iv, every day for 10 days (Monday through Friday).

FIG. 2: (A) Graph of therapeutic synergy observed with the combination of CA4P and Cisplatin in the M5 076DDP tumor model. Drug treatment was iv, Q4D×3. Drug combinations were administered simultaneously. (B) Graph showing CA4P significantly enhanced the antitumor activity of an otherwise inactive dose of cisplatin (3 mg/kg/inj).

FIG. 3: (A) Graph of therapeutic synergy observed with the combination of CA4P and Carboplatin in the M507 6 murine fibrosarcoma model. Drug treatment was intraperitoneal (ip), Q4D×3. Drug combinations were administered simultaneously ip (admixed). (B) Graph showing that CA4P, at three different dose levels (90-250 mg/kg/inj), significantly improved the antitumor activity of carboplatin.

FIG. 4: A graph showing antitumor activity in log cell kill indicating that the CA4P and carboplatin may be administered essentially simultaneously to achieve a potentiated therapeutic effect.

FIG. 5: Graph of inhibition of tumor blood flow by CA4P in the sc A27 80 human ovarian carcinoma grown in nude mice (A) or nude rats (B).

FIG. 6: Graph showing the antitumor effects of combining CPT-11 and CA4P chemotherapy in human ovarian carcinoma cells (A2780). CPT-11 is administered 3-24 hours prior to the administration of the combretastatin compound.

FIG. 7: Enhancement of the antitumor efficacy of carboplatin by low dose CA4P in M5076/DDP tumors. Panels A-C depict results for the combination of various doses of CA4P with 90, 60 and 40 mg/m² of carboplatin, respectively.

FIG. 8: Graph showing the synergistic antitumor efficacy obtained with a combination of paclitaxel and CA4P in a CaNT murine adenocarcinoma model. CA4P/Taxol combination therapy resulted in a considerable improvement in tumor growth delay as compared to a single dose (i.p.) of either CA4P (100 mg/kg) or Paclitaxel (30 mg/kg) alone. CA4P/Taxol combination therapy comprised a single dose of CA4P (100 mg/kg) followed 15 minutes later by a single dose of Paclitaxel (30 mg/kg).

FIGS. 9A and 9B: A pair of graphs showing that CA1P inhibits blood flow in human tumor xenografts in nude mice in a manner comparable to that observed for CA4P. FIG. 9A: N87 gastric cancer xenograft model; FIG. 9B: A2780 ovarian cancer xenograft model.

FIGS. 10A-10D: A series of graphs showing dose response curves of tumor size reduction in response to administration of CA1P and carboplatin alone and in combination against a M507 6 fibrosarcoma xenograft model. Combined administration of CA1P and carboplatin acted synergistically to reduce tumor size.

FIG. 11: Graph showing that combined administration of CA1P and carboplatin produces a synergistic antitumor effect. A complete response (disappearance of tumors) is observed with this combination.

FIG. 12: A graph showing that combined administration of cisplatin and CA1P act synergistically to reduce tumor size in a CaNT breast tumor model in CBA mice.

FIG. 13: A graph showing that CA4P potentiates the anti-tumor activity of Paclitaxel/Carboplatin two-agent chemotherapy such that a synergistic tumor growth delay is achieved in an ES-2 multidrug resistant ovarian tumor model in mice.

FIG. 14: A graph showing that the combined administration of carboplatin, paclitaxel, and CA4P is highly effective in increasing survival of mice bearing an ES-2 ovarian tumor model that is resistant to treatment with both paclitaxel and carboplatin.

FIG. 15: A graph showing that the combined administration of carboplatin, paclitaxel, and CA1P is highly effective in reducing tumor volume in mice bearing an ES-2 ovarian tumor model that is resistant to treatment with both paclitaxel and carboplatin.

FIG. 16: A graph showing that the combined administration of CA1P, Carboplatin, and Paclitaxel lead to significant improvement in the survival of mice bearing a bearing a ES-2 multidrug resistant ovarian tumor model.

FIG. 17: A graph showing that the combined administration of Carboplatin, and Paclitaxel, together with either CA1P or CA4P, leads to enhanced tumor growth delay in mice bearing a MDA-MB-234 human breast xenograft model, regardless of the sequence of administration.

FIG. 18: A graph showing that a significant increase in the number of neutrophils and a significant decrease in the number of lymphocytes is observed in tumor tissue at 4 hours following treatment with CA4P.

FIG. 19: A graph showing that significant increase in neutrophil to lymphocyte ratio is observed in tumor tissue at 4 hours following treatment with CA4P.

### SUMMARY OF THE INVENTION

The present invention provides effective methods for producing an antitumor effect wherein a combination of agents is employed. The methods of the present invention provide advantages such as greater overall efficacy, for example, in achieving synergy or avoiding antagonism, and allow, where desired, a reduction in the amount of one or more of the individual agents employed with a concomitant reduction in side effects. Further, where the tumor to be treated is not optimally responsive (e.g. resistant) to a given anticancer agent, use of the present combination therapy methods can nonetheless provide effective treatment.

In one aspect, the invention provides a method for producing an anti-tumor effect in a patient suffering from a cancer or tumor, the method comprising administering to the patient at least two anticancer agents and a combretastatin compound in amounts effective therefore. In one embodiment one of the at least two anticancer agents is a taxane. In another embodiment, one of the at least two anticancer agents is a platinum coordination compound. In a preferred embodiment, two of the anticancer agents are a taxane and a platinum coordination compound. Particularly preferred taxanes and platinum coordination compounds are paclitaxel and carboplatin respectively. Preferred combretastatin compounds are selected from the group consisting of CA1, CA4, CA1P, CA4P, or a prodrug or salt thereof. In one embodiment, the resultant anti-tumor effect is a potentiation of the overall efficacy of said other anticancer agents when used either alone or in a combination comprising two or more said other anticancer agents. The combretastatin compound may be administered at any time relative to administration of said other anticancer agents. In one embodiment, the combretastatin and the at least two other anticancer agents may be administered simultaneously to produce a potentiated antitumor effect. In another embodiment the combretastatin and the at least two other anticancer agents may be administered sequentially in any order to produce a potentiated antitumor effect. In one preferred embodiment, a combretastatin compound is sequentially administered in any order with effective amounts of a taxane and a platinum coordination compound. In a preferred embodiment, CA4P is sequentially administered in any order with an effective amount of a taxane and a platinum coordination compound. In a still more preferred embodiment, CA4P or CA1P are sequentially or simultaneously administered in any order with an effective amount of paclitaxel and carboplatin.

In another aspect, the invention provides a method for producing an anti-tumor effect in patient bearing a tumor, particularly a solid tumor, that is not optimally responsive (e.g. refractive or resistant) to treatment with one or more anticancer agents, comprising administering to the patient the one or more anticancer agents together with a combretastatin, in amounts effective to achieve an antitumor effect. In one embodiment, the tumor comprises cells that have acquired resistance to the one or more anticancer agents. In one exemplary embodiment, the solid tumor comprises cells that have acquired resistance to a taxane. In another exemplary embodiment, the solid tumor comprises cells that have acquired resistance to a platinum coordination compound. In a specific exemplary embodiment, the solid tumor has acquired resistance to both carboplatin and paclitaxel. In another embodiment, the solid tumor comprises cells that are resistant to treatment with a combretastatin.

In another aspect, the invention provides methods for determining the clinical prognosis of a patient suffering from cancer, wherein said patient has been administered an anticancer agent, the method comprising: (a) obtaining a biological sample from the patient; (b) determining a granulocyte level of the biological sample; (c) comparing the granulocyte level with a baseline level; (d) correlating the granulocyte level with an indication of unfavorable prognosis if the granulocyte level is greater than the baseline level or correlating the neutrophil level with an indication of favorable prognosis if the granulocyte level is equal to or less than the baseline. Preferably said anti-cancer agent is a combretastatin. In another embodiment, the granulocyte is a neutrophil. In another embodiment, the biological sample is obtained less than 24 hours after treatment with the anti-cancer agent. In a more preferred embodiment, the biological sample is obtained less than 6 hours after treatment with the anti-cancer agent.

In another aspect, the invention provides methods for selecting a patient for further treatment with an anti-cancer agent, the method comprising: (a) determining a granulocyte level in a first biological sample from the patient; (b) administering the anti-cancer agent to the patient; (c) determining a second granulocyte level from a second biological sample obtained from the patient; (d) comparing the first and second granulocyte levels; and (e) selecting the patient for further treatment if an increase in granulocyte level is observed.

In another aspect, the invention provides a method for monitoring the progression of a tumor in patient, the method comprising: (a) determining a granulocyte level in a first biological sample from the patient; (b) administering the anti-cancer agent to the patient; (c) determining a second granulocyte level from a second biological sample obtained from the patient; and (d) comparing the first and second granulocyte levels.

In another aspect, the present invention also provides pharmaceutical compositions comprising at least two other anticancer agents and a combretastatin compound. For example, in one aspect, the at least two other anticancer agents and/or combretastatin compound can be present in a subtherapeutic dose for the individual agent, the agents being more effective when used in combination or providing reduced side effects while maintaining efficacy. Alternatively, each agent can be provided at higher doses for the individual agent, such as those found in the Physician's Desk Reference.

In another aspect, the present invention further provides pharmaceutical kits. Exemplary kits of the invention comprise a first pharmaceutical composition comprising a first anticancer agent and a second pharmaceutical composition comprising a combretastatin compound together in a package. The anticancer agent and/or combretastatin compound can be present, for example, in a subtherapeutic dose for the individual agent, the agents being effective in combination and providing reduced side effects while maintaining efficacy. Alternatively, each agent can be provided at a higher dose, such as those found for the agent in the Physician's Desk Reference.

In certain aspects, the present invention provides sequences of administering a combretastatin and the one or more other anticancer agents to potentiate the overall efficacy of the combination. Combretastatin compounds, as antivascular agents, modulate blood flow to tumor tissue. By timing the administration of the combretastatin compound to modulate the flow of blood to the tumor it is possible to provide a time-dependent effective tumor concentration of the other anticancer agent such that the overall efficacy of the combination is potentiated.

The present invention therefore provides, as a further embodiment, a method for modulating tumor growth or metastasis in an animal in need thereof, especially a human, comprising administration of a combretastatin compound and at least one anticancer agent, in amounts effective therefor, wherein said combretastatin is administered at a time relative to administration of said anticancer agent sufficient to modulate blood flow to said tumor to provide a time-dependent effective tumor concentration of said anticancer agent. The method of the present invention allows potentiation of the overall efficacy of the combination employed.

In one embodiment, Peak Tumor Concentration Agents, such as platinum based anticancer agents, including cisplatin or carboplatin, are administered sequentially in any order with a combretastatin compound, such as a CA4P compound or a CA1P compound. In a preferred embodiment, Peak Tumor Concentration Agents, such as platinum based anticancer agents, including cisplatin or carboplatin, are administered essentially simultaneously with a combretastatin compound, such as CA4P or CA1P.

In yet another embodiment, Duration Exposure Agents, including immunotoxins, and taxanes, such as paclitaxel and docetaxel are administered sequentially, in any order, with a combretastatin compound. In a preferred embodiment, the Duration Exposure Agents are administered prior to the Duration Exposure Agent to extend the exposure time of the tumor tissue to the Duration Exposure Agent.

In an additional embodiment, High AUC Agents such as CPT-11 are administered sequentially in any order prior to the administration of a combretastatin compound (e.g., CA4P or CA1P). In an additional preferred embodiment, High AUC Agents are administered prior to the administration of the combretastatin compound.

When administered sequentially with a combretastatin, such agents can preferably be administered, for example, within 24 hours of the administration of the combretastatin compound, such as within 1-24 hours prior, 2-24 hours prior, 3-24 hours prior, 6-24 hours prior, 8-24 hours prior, or 12 to 24 hours prior to administration.

The present invention further provides chemotherapeutic pharmaceutical compositions comprising both a combretastatin compound, and at least one selected anticancer agent and the use thereof in the present methods. Alternatively, the method of the present invention can be carried out using chemotherapeutic pharmaceutical compositions, which comprise one of the above-described compounds as the active ingredient, in combination with a pharmaceutically acceptable carrier medium or an auxiliary agent. Thus, in such an embodiment, the combretastatin compound, such as CA4P or CA1P, and the anticancer agent, such as cisplatin are formulated and administered separately.

### DETAILED DESCRIPTION OF THE INVENTION

Derived from the South African tree Combretum caffrum, combretastatins such as Combretastatin A-4 (CA-4) were initially identified in the 1980's as potent inhibitors of tubulin polymerization. CA-4, and other combretastatins (e.g. CA-1) have been shown to bind a site at or near the colchicine binding site on tubulin with high affinity. In vitro studies clearly demonstrated that combretastatins are potent cytotoxic agents against a diverse spectrum of tumor cell types in culture. CA4P and CA1P, respective phosphate prodrugs of CA-4 and CA-1, were subsequently developed to combat problems with aqueous insolubility. Surprisingly, CA1P and CA4P have also been shown to cause a rapid and acute shutdown of the blood flow to tumor tissue that is separate and distinct from the anti-proliferative effects of the agents on tumor cells themselves. A number of studies have shown that combretastatins cause extensive shut-down of blood flow within the tumor microvasculature, leading to secondary tumor cell death (Dark et al., Cancer Res., 57: 1829-34, (1997); Chaplin et al., Anticancer Res., 19: 189-96, (1999); Hill et al., Anticancer Res., 22(3):1453-8 (2002); Holwell et al., Anticancer Res., 22(2A):707-11, (2002). Blood flow to normal tissues is generally far less affected by CA4P and CA1P than blood flow to tumors, although blood flow to some organs, such as spleen, skin, skeletal muscle and brain, can be inhibited (Tozer et al., Cancer Res., 59: 1626-34 (1999)).

In light of the novel, non-cytotoxic, mode of action of combretastatins, there is considerable interest in exploiting the novel “vascular targeting” of these agents for cancer treatment. Recently, single agent efficacy was reported for CA4P using a frequent dosing regimen. Another report suggested that large tumors can, in some cases, be more responsive to CA4P therapy than small tumors. However, many tumors harvested from animals treated with CA4P reveal central necrosis surrounded by a rim of viable cells (Dark et al., Cancer Res., 57: 1829-34, (1997); Chaplin et al., Anticancer Res., 19: 189-96, (1999)). This rim of surviving cells is most likely a consequence of the shared normal vessel circulation between the perimeter of tumors and neighboring normal tissue. The inventors have made the surprising discovery that combining them with cytotoxic agents can optimize antitumor activity of combretastatins.

In another aspect, the inventors have found that the novel mechanism of action of combretastatins make it them ideal agents for combination chemotherapy. The inventors have found that when combined with conventional chemotherapy, combretastatins such as CA4P or CA1P compounds can potentiate the activity of the conventional chemotherapeutics. Moreover, combretastatins have been found to have different toxicities, which do not overlap with those of conventional chemotherapeutics. This property allows for the highest effective dose of each agent to be used in the combination, rather than having to reduce the dose of one or both agents to compensate for an overlapping toxicity. Finally, and perhaps most surprisingly, the inventors have found that combretastatins are effective in treating many cancers that are resistant to conventional chemotherapeutics.

a) Definitions

As used herein, the term “combretastatin” denotes at least one of combretastatin family of compounds, derivatives or analogs thereof, their prodrugs (preferably phosphate prodrugs) and derivatives thereof, and salts of these compounds. Combretastatins include those anti-cancer compounds isolated from the South African tree Combretum caffrum, including without limitation, Combretastatins A-1, A-2, A-3, A-4, B-1, B-2, B-3, B-4, D-1, and D-2, and various prodrugs thereof, exemplified by Combretastatin A-4 phosphate (CA4P) compounds, Combretastatin A-1 diphosphate (CA1P) compounds and salts thereof (see for example Pettit et al, Can. J. Chem., (1982); Pettit et al., J. Org. Chem., 1985; Pettit et al., J. Nat. Prod., 1987; Lin et al., Biochemistry, (1989); Pettit et al., J. Med. Chem., 1995; Pettit et al., Anticancer Drug Design, (2000); Pettit et al., Anticancer Drug Design, 16(4-5): 185-93 (2001)).

Combretastatin salts contemplated for use in the methods of the invention are described in WO 99/35150; WO 01/81355; U.S. Pat. Nos. 6,670,344; 6,538,038; 5,569,786; 5,561,122; 5,409,953; 4,996,237 which are incorporated herein by reference in their entirety. Preferred CA4P compounds are disodium salts or those of the formula I: 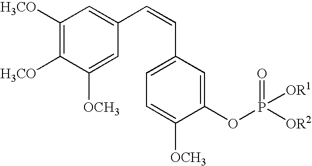
wherein one of OR¹ and OR² is —O⁻QH⁺ or —O⁻M⁺ and the other is hydroxyl, —O⁻QH⁺, or —O⁻M⁺, and
wherein M⁺ is a monovalent or divalent metal cation (e.g. Na⁺, K⁺, Mg²⁺) and Q is:

a) an amino acid containing at least two nitrogen atoms where one of the nitrogen atoms, together with a proton, forms a quaternary ammonium cation QH⁺, preferably, where one of OR¹ and OR² is hydroxyl, and the other is —O⁻QH⁺ where Q is L-histidine; or

b) an organic amine wherein one of OR¹ and OR² is —O⁻QH⁺, and the other is hydroxyl or —O⁻QH⁺; and Q is an organic amine containing at least one nitrogen atom which, together with a proton, forms a quaternary ammonium cation, QH⁺, preferably, where one of OR¹ and OR² is hydroxyl and the other is —O⁻QH⁺ and Q is tris(hydroxymethyl)amino methane (“TRIS”).

As used herein, the term combretastatin A-1 diphosphate (CA1P) compound denotes as least one of combretastatin A-1 diphosphate prodrugs derivatives thereof, and salts of these compounds. A preferred CA1P compound has the following general structure: 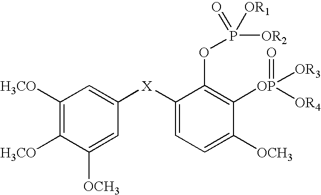
wherein X is a carbon-carbon double bond in the cis configuration and at least one of OR¹, OR², OR³, and OR⁴ is —O⁻QH⁺ or —O⁻M⁺ and the other is hydroxyl, —O⁻QH⁺, or —O⁻M⁺, and
wherein M⁺ is a monovalent or divalent metal cation (e.g. Na⁺, K⁺, Mg²⁺) and Q is:

a) an amino acid containing at least two nitrogen atoms where one of the nitrogen atoms, together with a proton, forms a quaternary ammonium cation QH⁺, preferably, where one of OR¹ and OR² is hydroxyl, and the other is —O⁻QH⁺ where Q is L-histidine; or

b) an organic amine wherein one of OR¹ and OR² is —O⁻QH⁺, and the other is hydroxyl or —O⁻QH⁺; and Q is an organic amine containing at least one nitrogen atom which, together with a proton, forms a quaternary ammonium cation, QH⁺, preferably, where one of OR¹ and OR² is hydroxyl and the other is —O⁻QH⁺ and Q is tris(hydroxymethyl)amino methane (“TRIS”).

Derivatives or analogs of combretastatins are described in Singh et al., J. Org. Chem., 1989; Cushman et al, J. Med. Chem., 1991; Getahun et al, J. Med. Chem., 1992; Andres et al, Bioorg. Med. Chem. Lett., 1993; Mannila, et al., Liebigs. Ann. Chem., 1993; Shirai et al., Bioorg. Med. Chem. Lett., 1994; Medarde et al., Bioorg. Med. Chem. Lett., 1995; Wood et al, Br. J. Cancer, 1995; Bedford et al., Bioorg. Med. Chem. Lett., 1996; Dorr et al., Invest. New Drugs, 1996; Jonnalagadda et al., Bioorg. Med. Chem. Lett., 1996; Shirai et al., Heterocycles, 1997; Aleksandrzak, et al., Anticancer Drugs, 1998; Chen et al., Biochem. Pharmacol., 1998; Ducki et al., Bioorg. Med. Chem. Lett., 1998; Hatanaka et al., Bioorg. Med. Chem. Lett., 1998; Medarde et al., Eur. J. Med. Chem., 1998; Medina et al., Bioorg. Med. Chem. Lett., 1998; Ohsumi et al., Bioorg. Med. Chem. Lett., 1998; Ohsumi et al., J. Med. Chem., 1998; Pettit, et al., J. Med. Chem., 1998; Shirai et al., Bioorg. Med. Chem. Lett., 1998; Banwell et al., Aust. J. Chem., 1999; Medarde et al., Bioorg. Med. Chem. Lett., 1999; Shan et al., PNAS, 1999; Combeau et al., Mol. Pharmacol., 2000; Pettit et al., J. Med. Chem., 2000; Pinney et al., Bioorg. Med. Chem. Lett., 2000; Flynn et al., Bioorg. Med. Chem. Lett., 2001; Gwaltney et al., Bioorg. Med. Chem. Lett., 2001; Lawrence et al., 2001; Nguyen-Hai et al., Bioorg. Med. Chem. Lett., 2001; Xia et al., J. Med. Chem., 2001; Tahir et al., Cancer Res., 2001; Wu-Wong et al., Cancer Res., 2001; Janik et al, Biooorg. Med. Chem. Lett., 2002; Kim et al., Bioorg Med Chem Lett., 2002; Li et al., Biooorg. Med. Chem. Lett., 2002; Nam et al., Bioorg. Med. Chem. Lett., 2002; Wang et al., J. Med. Chem. 2002; Hsieh et al., Biooorg. Med. Chem. Lett., 2003; Hadimani et al., Bioorg. Med. Chem. Lett., 2003; Mu et al., J. Med. Chem, 2003; Nam et al., Curr. Med. Chem., 2003; Pettit et al, J. Med. Chem., 2003; WO 03/040077, WO 03/035008, WO 02/50007, WO 02/14329; WO 01/12579, WO 01/09103, WO 01/81288, WO 01/84929, WO 00/48590, WO 00/73264, WO 00/06556, WO 00/35865, WO 99/34788, WO 99/48495, WO 92/16486, U.S. Pat. Nos. 6,794,384; 6,787,672, 6,777,578, 6,723,858, 6,720,323, 6,433,012, 6,423,753, 6,201,001, 6,150,407, 6,169,104, 5,731,353, 5,674,906, 5,430,062, 5,525,632, 4,996,237 and 4,940,726 and U.S. patent application Ser. No. 10/281,528; and U.S. patent application Ser. No. 10/281,528, which are incorporated herein by reference in their entirety.

As used herein, “paclitaxel” refers to paclitaxel and analogues and derivatives thereof, including, for example, a natural or synthetic functional variant of paclitaxel, which has paclitaxel biological activity, as well as a fragment of paclitaxel having paclitaxel biological activity. As further used herein, the term “paclitaxel biological activity” refers to paclitaxel activity, which interferes with cellular mitosis by affecting microtubule formation and/or action, thereby producing antimitotic and antineoplastic effects. Methods of preparing paclitaxel and its analogues and derivatives are well known in the art, and are described, for example, in U.S. Pat. Nos. 5,569,729; 5,565,478; 5,530,020; 5,527,924; 5,484,809; 5,475,120; 5,440,057; and 5,296,506. Paclitaxel and its analogues and derivatives are also available commercially. Synthetic paclitaxel, for example, can be obtained from Bristol-Myers Squibb Company, Oncology Division (Princeton, N.J.), under the registered trademark Taxol®. Taxol for injection may be obtained in a single-dose vial, having a concentration of 30 mg/5 mL (6 mg/mL per 5 mL). For example, doses of paclitaxel (Taxol) administered intraperitoneally may be between 1 and 10 mg/kg, and doses administered intravenously may be between 1 and 3 mg/kg, or between 135 mg/m² and 200 mg/m². However, the amounts of paclitaxel and discodermolide effective to treat neoplasia in a subject in need of treatment will vary depending on the particular factors of each case, including the type of neoplasm, the stage of neoplasia, the subject's weight, the severity of the subject's condition, and the method of administration. The skilled artisan can readily determine these amounts.

Platinum coordination compounds as defined herein include anticancer alkylating agents, which produce predominantly interstrand DNA cross-links. Preferred platinum coordination compounds include Carboplatin, Cisplatin, and Oxaliplatin. Carboplatin is commercially available for intravenous injection under the registered trademark Paraplatin® (Bristol Myers Squibb, Princeton, N.J.).

As used herein, the term “effective amount” of a compound or pharmaceutical composition refers to an amount sufficient to provide the desired anti-cancer effect or anti-tumor effect in an animal, preferably a human, suffering from cancer. Desired anti-tumor effects include, without limitation, the modulation of tumor growth (e.g. tumor growth delay), tumor size, or metastasis, the reduction of toxicity and side effects associated with a particular anti-cancer agent, the amelioration or minimization of the clinical impairment or symptoms of cancer, extending the survival of the subject beyond that which would otherwise be expected in the absence of such treatment, and the prevention of tumor growth in an animal lacking any tumor formation prior to administration, i.e., prophylactic administration. As used herein, the terms “modulate”, “modulating” or “modulation” refer to changing the rate at which a particular process occurs, inhibiting a particular process, reversing a particular process, and/or preventing the initiation of a particular process. Accordingly, if the particular process is tumor growth or metastasis, the term “modulation” includes, without limitation, decreasing the rate at which tumor growth and/or metastasis occurs; inhibiting tumor growth and/or metastasis; reversing tumor growth and/or metastasis (including tumor shrinkage and/or eradication) and/or preventing tumor growth and/or metastasis. “Synergistic effect”, as used herein refers to a greater-than-additive anti-cancer effect which is produced by a combination of two drugs, and which exceeds that which would otherwise result from individual administration of either drug alone. One measure of synergy between two drugs is the combination index (CI) method of Chou and Talalay (see Chang et al., Cancer Res. 45: 2434-2439, (1985)), which is based on the median-effect principle. This method calculates the degree of synergy, additivity, or antagonism between two drugs at various levels of cytotoxicity. Where the CI value is less than 1, there is synergy between the two drugs. Where the CI value is 1, there is an additive effect, but no synergistic effect. CI values greater than 1 indicate antagonism. The smaller the CI value, the greater the synergistic effect. Another measurement of synergy is the fractional inhibitory concentration (FIC). This fractional value is determined by expressing the IC₅₀ of a drug acting in combination, as a function of the IC₅₀ of the drug acting alone. For two interacting drugs, the sum of the FIC value for each drug represents the measure of synergistic interaction. Where the FIC is less than 1, there is synergy between the two drugs. An FIC value of 1 indicates an additive effect. The smaller the FIC value, the greater the synergistic interaction.

The term “anticancer agent” as used herein denotes a chemical compound or electromagnetic radiation (especially, X-rays), which is capable of modulating tumor growth or metastasis. When referring to use of such an agent with a combretastatin compound, the term refers to an agent other than a combretastatin compound. Unless otherwise indicated, this term can include one, or more than one, such agents. Thus, the term “anticancer agent” encompasses the use of one or more chemical compounds and/or electromagnetic radiation in the present methods and compositions. Where more than one anticancer agent is employed, the relative time for administration of the combretastatin compound can, as desired, be selected to provide a time-dependent effective tumor concentration of one, or more than one, of the anticancer agents.

As explained above, numerous types of anticancer agents are exemplary of those having applications in a composition or method of the present invention. Such classes of anticancer agents, and their preferred mechanisms of action, are described below:

1. Alkylating agent: a compound that donates an alkyl group to nucleotides. Alkylated DNA is unable to replicate itself and cell proliferation is stopped. Examples of such compounds include, but are not limited to, busulfan, coordination metal complexes (e.g. platinum coordination compounds such as carboplatin, oxaliplatin, and cisplatin), cyclophosphamide (cytoxan), dacarbazine, ifosfamide, mechlorethamine (mustargen), and melphalan;

2. Bifunctional alkylating agent: a compound having two labile methanesulfonate groups that are attached to opposite ends of a four carbon alkyl chain. The methanesulfonate groups interact with, and cause damage to DNA in cancer cells, preventing their replication. Examples of such compounds include, without limitation, chlorambucil and melphalan;

3. Non-steroidal aromatase inhibitor: a compound that inhibits the enzyme aromatase, which is involved in estrogen production. Thus, blockage of aromatase results in the prevention of the production of estrogen. Examples of such compounds include anastrozole and exemstane;

4. Immunotherapeutic agent: an antibody or antibody fragment that targets cancer cells that produce proteins associated with malignancy. Exemplary immunotherapeutic agents include Herceptin which targets HER2 or HER2/neu, which occurs in high numbers in about 25 percent to 30 percent of breast cancers; Erbitux which targets the Epidermal Growth Factor Receptor (EGFR) in colon cancers; Avastin which targets the Vascular Endothelial Growth Factor (VEGF) expressed by colon cancers; and Rituxan an anti-CD20 which triggers apoptosis in B cell lymphomas. Additional immunotherapeutic agents include immunotoxins, wherein toxin molecules such as ricin, diphtheria toxin and pseudomonas toxins are conjugated to antibodies, which recognize tumor specific antigens. Conjugation can be achieved biochemically or via recombinant DNA methods.

5. Nitrosurea compound: inhibits enzymes that are needed for DNA repair. These agents are able to travel to the brain so they are used to treat brain tumors, as well as non-Hodgkin's lymphomas, multiple myeloma, and malignant melanoma. Examples of nitrosureas include carmustine and lomustine;

6. Antimetabolite: a class of drugs that interfere with DNA and ribonucleic acid (RNA) synthesis. These agents are phase specific (S phase) and are used to treat chronic leukemias as well as tumors of breast, ovary and the gastrointestinal tract. Examples of antimetabolites include 5-fluorouracil, methotrexate, gemcitabine (GEMZAR®), cytarabine (Ara-C), and fludarabine.

7. Antitumor antibiotic: a compound having antimicrobial and cytotoxic activity. Such compounds also may interfere with DNA by chemically inhibiting enzymes and mitosis or altering cellular membranes. Examples include, but certainly are not limited to bleomycin, dactinomycin, daunorubicin, doxorubicin (Adriamycin), idarubicin, and the manumycins (e.g. Manumycins A, C, D, E, and G and their derivatives; see for example U.S. Pat. No. 5,444,087);

8. Mitotic inhibitor: a compound that can inhibit mitosis (e.g., tubulin binding compounds) or inhibit enzymes that prevent protein synthesis needed for reproduction of the cell. Examples of mitotic inhibitors include taxanes such as paclitaxel and docetaxel, epothilones, etoposide, vinblastine, vincristine, and vinorelbine.

9. Radiation therapy: includes but is not limited to X-rays or gamma rays which are delivered from either an externally supplied source such as a beam or by implantation of small radioactive sources.

10. Topoisomerase I inhibitors: agents, which interfere with topoisomerase activity thereby inhibiting DNA replication. Such agents include, without limitation, CPT-11 and topotecan.

11. Hormonal therapy: includes, but is not limited to anti-estrogens, such as Tamoxifen, GNRH agonists, such as Lupron, and Progestin agents, such as Megace.

Naturally, other types of anticancer agents that function via a large variety of mechanisms have application in the pharmaceutical compositions and methods of the present invention. Additional such agents include for example, leucovorin, kinase inhibitors, such as Iressa and Flavopiridol, analogues of conventional chemotherapeutic agents such as taxane analogs and epothilone analogues, antiangiogenics such as matrix metalloproteinase inhibitors, and other VEGF inhibitors, such as ZD6474 and SU6668. Retinoids such as Targretin can also be employed in the pharmaceutical compositions and methods of the invention. Signal transduction inhibitors that interfere with famesyl transferase activity and chemotherapy resistance modulators, e.g., Valspodar can also be employed. Monoclonal antibodies such as C225 and anti-VEGFr antibodies can also be employed.

As used herein, the term “prodrug” refers to a precursor form of the drug, which is metabolically converted in vivo to produce the active drug. Thus, for example, combretastatin phosphate prodrug salts administered to an animal in accordance with the present invention undergo metabolic activation and regenerate combretastatin A-4 or combretastatin A-1 in vivo, e.g., following dissociation and exposure to endogenous non-specific phosphatases in the body.

As explained above, the present invention is directed towards a pharmaceutical composition that modulates growth or metastasis of tumors, particularly solid tumors, using a pharmaceutical composition of the present invention, along with methods of modulating tumor growth or metastasis, for example, with a pharmaceutical composition of the present invention.

As used herein, the terms “tumor”, “tumor growth” or “tumor tissue” can be used interchangeably, and refer to an abnormal growth of tissue resulting from uncontrolled progressive multiplication of cells and serving no physiological function. A solid tumor can be malignant, e.g. tending to metastasize and being life threatening, or benign. Examples of solid tumors that can be treated or prevented according to a method of the present invention include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, gastric cancer, pancreatic cancer, breast cancer, ovarian cancer, fallopian tube cancer, primary carcinoma of the peritoneum, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, liver metastases, bile duct carcinoma, choriocarcinoma, seminoma, embryonal carcinoma, thyroid carcinoma such as anaplastic thyroid cancer, Wilms' tumor, cervical cancer, testicular tumor, lung carcinoma such as small cell lung carcinoma and non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Moreover, tumors comprising dysproliferative changes (such as metaplasias and dysplasias) can be treated or prevented with a pharmaceutical composition or method of the present invention in epithelial tissues such as those in the cervix, esophagus, and lung. Thus, the present invention provides for treatment of conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68 to 79). Hyperplasia is a form of controlled cell proliferation involving an increase in cell number in a tissue or organ, without significant alteration in structure or function. For example, endometrial hyperplasia often precedes endometrial cancer. Metaplasia is a form of controlled cell growth in which one type of adult or fully differentiated cell substitutes for another type of adult cell. Metaplasia can occur in epithelial or connective tissue cells. Atypical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder. For a review of such disorders, see Fishman et al., 1985, Medicine, 2d Ed., J. B. Lippincott Co., Philadelphia.

Other examples of tumors that are benign and can be treated or prevented in accordance with a method of the present invention include arteriovenous (AV) malformations, particularly in intracranial sites and myelomas.

The term “time-dependent effective tumor concentration,” as used herein, denotes a concentration of the other anticancer agent in the tumor tissue over time (i.e., from administration until the agent is cleared from the body) that potentiates the action of the combination of the combretastatin compound and other anticancer agent.

The phrase “Peak Tumor Concentration Agents” refers to anticancer agents, which are most efficacious at high tumor concentrations yet are rapidly cleared from the tumor tissue. Such agents may be administered simultaneously with or in close temporal proximity to (e.g., as is clinically feasible, especially within one hour of) the administration of the combretastatin compound in accordance with the invention. Exemplary Peak Tumor Concentration Agents include, without limitation, alkylating agents (e.g. cytoxan and mitomycin C) and metal coordination complexes such as cisplatin, oxaliplatin and carboplatin.

The phrase “Duration Exposure Agents” as used herein refers to agents which can be effective at relatively low tumor concentrations yet which require certain tumor tissue exposure times to be most effective. Such agents may be administered sequentially in any order with a combretastatin compound in accordance with the invention, provided that a sufficient delay is allowed between administrations to potentiate the combination. In one embodiment of the method of the invention, the Duration Exposure Agent is administered after the administration of the combretastatin A-4 compound or combretastatin A-1 compound. Exemplary Duration Exposure Agents include, without limitation, taxanes such as paclitaxel and docetaxel, etoposide, etoposide phosphate, immunotoxins, and epothilones.

The phrase “High AUC Agents” as used herein refers to those agents, which show greatest efficacy when present at high concentrations in tumor tissue for extended time periods. Such agents are may be administered sequentially with a combretastatin compound in accordance with the invention, wherein the High AUC Agent is administered first, followed by the combretastatin compound, provided that a sufficient delay is allowed between administrations to potentiate the combination. Exemplary High AUC Agents include, without limitation, adriamycin, CPT-11 (irinotecan), and topotecan.

As used herein, the term “biological sample” includes, for example, a sample of blood, tissue (e.g. tumor tissue), serum, stool, urine, sputum, cerebrospinal fluid, and cell supematant from a cell lysate. In an exemplary embodiment, blood is employed as the biological sample in the methods of the invention.

b) Preferred Dosage Ranges—Two-Component Combination Therapy

In accordance with the present invention, improved, two-component chemotherapeutic regimens comprising a combretastatin and an anticancer agent are provided for the treatment of cancer. The improved chemotherapeutic regimens can lower side effects and enhance efficacy for the treatment of neoplastic disease. Surprisingly, the two-component combinations overcome many of the disadvantages single anti-cancer agent therapy (i.e. monotherapy). For example, the present methods permit a clinician to administer a combretastatin compound, such as CA4P or CA1P, and/or an anticancer agent, at dosages that are significantly lower than those employed for the single agent. Preferred dosages suitable for administration of the anticancer agent and combretastatin compounds in accordance with the invention are set forth herein below. Whether administered simultaneously or sequentially, the combretastatin compound and the at least one anticancer agent can be administered in any amount or by any route of administration effective for the modulation of tumor growth or metastasis, especially treatment of cancer as described herein.

The following Table I sets forth preferred two-component chemotherapeutic combinations and exemplary dosages for use in the methods of the present invention. Where “Combretastatin” appears, combretastatin A-4, combretastatin A-1 or a phosphate prodrug of either combretastatin A-4 or combretastatin A-1 or, such as CA4P or CA1P, is preferably employed.

TABLE 1
Exemplary Two-Component Combination
Therapies and Dosage Range

| | Dosage | |
|---|---|---|
| Chemotherapeutic combination | mg/m² (per dose) | |
| Combretastatin + | 1-100 | mg/m2 |
| Cisplatin | 5-150 | mg/m2 |
| Combretastatin + | 1-100 | mg/m2 |
| Carboplatin | 5-1000 | mg/m2 |
| Combretastatin + | 1-100 | mg/m2 |
| Radiation | 200-8000 | cGy |
| Combretastatin + | 1-100 | mg/m2 |
| CPT-11 | 5-400 | mg/m2 |
| Combretastatin + | 1-100 | mg/m2 |
| Paclitaxel | 40-250 | mg/m2 |
| Combretastatin + | 1-100 | mg/m2 |
| Epothilone | 1-500 | mg/m2 |
| Combretastatin + | 1-100 | mg/m2 |
| Gemcitabine | 100-3000 | mg/m2 |
| Combretastatin + | 1-100 | mg/m2 |
| BR96-sFv-PE40 | 100-750 | mg/m2 |

In the above Table I, “5FU” denotes 5-fluorouracil, “Leucovorin” can be employed as leucovorin calcium, “UFT” is a 1:4 molar ratio of tegafur:uracil, and “Epothilone” is preferably a compound described in WO 99/02514 or WO 00/50423, both incorporated by reference herein in their entirety.

While Table I provides exemplary dosage ranges of CA4P and certain anticancer agents of the invention, when formulating the pharmaceutical compositions of the invention the clinician may utilize preferred dosages as warranted by the condition of the patient being treated. For example, combretastatin compounds may preferably be administered at a dosage ranging from 30-70 mg/M² every three weeks for as long as treatment is required. Preferred dosages for cisplatin are 75-120 mg/m² administered every three weeks. Preferred dosages for carboplatin are within the range of 200-600 mg/m² or an AUC of 0.5-8 mg/ml×min; most preferred is an AUC of 4-6 mg/ml×min. When the method employed utilizes radiation, preferred dosages are within the range of 200-6000 cGY. Preferred dosages for CPT-11 are within 100-125 mg/m², once a week. Preferred dosages for paclitaxel are 130-225 mg/m² every 21 days. Preferred dosages for gemcitabine are within the range of 80-1500 mg/m² administered weekly. Preferably UFT is used within a range of 300-400 mg/m² per day when combined with leucovorin administration. Preferred dosages for leucovorin are 10-600 mg/m² administered weekly. A preferred dose of the Br96-sFv-PE40 immunotoxin is 420 mg/m². The use of the BR96-sFv-PE40 immunotoxin in combination with combretastatin A4 and its prodrugs in immune enhancing therapy is described in U.S. Provisional Application 60/258,283, filed Dec. 26, 2000, the entire disclosure of which is incorporated by reference herein.

In one exemplary embodiment, a combretastatin prodrug (e.g. CA4P) is administered together with a taxane, preferably paclitaxel. Paclitaxel is a natural diterpene that has been isolated from several species of yew trees. It is also available commercially under the registered trademark Taxol® (Bristol-Myers Squibb, Princeton, N.J.). Both taxanes and combretastatins are antimitotic agents that bind tubulin. However, they have completely opposing and antagonistic mechanisms of action. Combretastatins bind tubulin monomers in a tumor and prevent their polymerization into microtubules, thereby effectively preventing the tumor cell from assembling a spindle apparatus to facilitate mitosis. Taxanes, on the other hand, enhance the assembly of microtubules from tubulin dimers, and stabilize them against depolymerization. This stability results in the inhibition of normal dynamic reorganization of the microtubule network that is essential for exit from mitosis. Paclitaxel is well-known as an effective antineoplastic chemotherapeutic agent. In fact, paclitaxel (Taxol®) has been used with success in the treatment of leukemias and tumors, particularly breast, lung, and ovarian carcinomas, and malignant melanoma (McGuire et al., N. Engld. J. Med. 334:1-6, 1996; Johnson et al., J. Clin. Ocol. 14:2054-2060, 1996). Despite its considerable clinical success, there are a number of serious disadvantages to the use of paclitaxel. One problem, for example, is related to severe side-effects it produces, including alopecia, arthralgia, myalgia, myelosuppression, and neuropathy. The inventors have discovered that, despite their antagonistic mechanism of action, CA4P may be administered sequentially in any order with a reduced dose level of paclitaxel to reduce toxicity and/or potentiate the efficacy of treatment. When administered together with a paclitaxel, CA4P is preferably used at a free acid dose ranging from 45-63 mg/m² one a week and paclitaxel is preferably administered within 24 hours of CA4P at a dose ranging from 135-175 mg/m²

c) Combination Therapy with Three or More Components—Preferred Dosage Ranges

Certain cancers can be treated effectively with combretastatin A-4 or combretastatin A-1 and a plurality of anticancer agents. Such triple and quadruple combinations can provide greater efficacy. When used in such triple and quadruple combinations the dosages set forth below in Table II can be utilized. Where “combretastatin” appears, combretastatin A-4, combretastatin A-1 or a phosphate prodrug of either combretastatin A-4 or combretastatin A-1 such as a CA4P or a CA1P compound, is preferably employed. Other such combinations than in Table II can therefore include a combretastatin in combination with (1) mitoxantrone+prednisone; (2) doxorubicin+taxane; or (3) herceptin+taxane. 5-FU can be replaced by UFT in any of the above combinations.

TABLE II
Exemplary Three-Component Combination
Therapies and Dosage Range

| | Dosage |
|---|---|
| Chemotherapeutic combination | mg/m² (per dose) |
| Combretastatin + Paclitaxel + | 1-100 |
| Carboplatin | 40-250 |
| | 5-1000 |
| Combretastatin + | 1-100 |
| 5FU and optionally + | 5-5000 |
| Leucovorin | 5-1000 |
| Combretastatin + | 1-100 |
| Epothilone | 1-500 |
| Combretastatin + | 1-100 |
| Gemcitabine | 100-3000 |
| Combretastatin + | 1-100 |
| UFT and optionally + | 50-800 |
| leucovorin | 5-1000 |
| Combretastatin + | 1-100 |
| Gemcitabine + | 100-3000 |
| Cisplatin | 5-150 |
| Combretastatin + | 1-100 |
| UFT + | 50-800 |
| Leucovorin | 5-1000 |
| Combretastatin + | 1-100 |
| Cisplatin + | 5-150 |
| paclitaxel | 40-250 |
| Combretastatin + | 1-100 |
| Cisplatin + | 5-150 |
| 5FU | 5-5000 |
| Combretastatin A-4 + | 1-100 |
| Oxaliplatin + | 5-200 |
| CPT-11 | 4-400 |
| Combretastatin A-4 + | 1-100 |
| 5FU + | 5-5000 |
| CPT-11 and optionally + | 4-400 |
| leucovorin | 5-1000 |
| Combretastatin A-4 + | 1-100 |
| 5FU + | 5-5000 |
| radiation | 200-8000 cGy |
| Combretastatin A-4 + | 1-100 |
| radiation + | 200-8000 cGy |
| 5FU + | 5-5000 |
| Cisplatin | 5-150 |
| Combretastatin A-4 + | 1-100 |
| Oxaliplatin + | 5-200 |
| 5FU and optionally + | 5-5000 |
| Leucovorin | 5-1000 |
| Combretastatin A-4 + | 1-100 |
| paclitaxel + | 40-250 |
| CPT-11 | 4-400 |
| Combretastatin A-4 + | 1-100 |
| paclitaxel + | 40-250 |
| 5FU | 5-5000 |
| Combretastatin A-4 + | 1-100 |
| paclitaxel + | 40-250 |
| manumycin A | 1-100 |
| Combretastatin A-4 + | 1-100 |
| UFT + | 50-800 |
| CPT-11 and optionally + | 4-400 |
| leucovorin | 5-1000 |

While Table II provides exemplary dosage ranges for certain anticancer agents of the invention, when formulating the pharmaceutical compositions of the invention the clinician may utilize preferred dosages as warranted by the condition of the patient being treated. For example, when administered together with carboplatin and paclitaxel in a three-component therapy, combretastatin compounds may preferably be administered at a dosage ranging from 27-70 mg/m² every three weeks for as long as treatment is required when administered. CA4P (free acid) is preferably administered at a dosage ranging from 27 mg/m² to 70 mg/m². More preferably CA4P (free acid) is administered at a dosage ranging from 45 mg/m² to 63 mg/m². Preferred dosages for carboplatin are within the range of 200-600 mg/m² or an AUC of 0.5-8 mg/ml×min; most preferred is an AUC of 4-6 mg/ml×min administered on the day following Combretastatin treatment. Preferred dosages for paclitaxel are within the range of 135-175 mg/m² administered on the day following CA4P treatment.

d) Treatment of Drug Resistant Tumors

Many of the most common carcinomas, including breast and ovarian cancer, are initially relatively sensitive to a wide variety anti-cancer agents. However, acquired drug resistance phenotype typically occurs after months or years of exposure to chemotherapy. Determining the molecular basis of drug resistance may offer opportunities for improved diagnostic and therapeutic strategies. Therefore, the present invention contemplates the treatment of patient suffering from a cancer or tumor which has demonstrated resistance to one or more anti-cancer agents, comprising administering to the patient a combretastatin, together with the one or more anti-cancer agents, in effective amounts to generate a potentiated response. The inventors have made the surprising discovery that a method of treatment comprising administering a combretastatin, together with one or more anticancer agents, is an effective method for treating solid tumors that are refractive or resistant to treatment with either a combretastatin alone or one or more anticancer agents. In particular, the inventors have discovered that the combination of an effective amount of a combretastatin, together with an effective amount of both a platinum coordination compound and a taxane, is effective in treating tumors that are resistant to treatment with the combretastatin alone or one or both of the anticancer agents.

Taxanes and the platinum coordination compounds (e.g., Carboplatin) have been shown to be effective when used individually for the treatment of some tumors. However, many tumors are nonetheless refractive to treatment regimes with these agents due to intrinsic or acquired resistance to one or both agents. It is known, for example, that a considerable number of patients initially responsive to treatment with taxane anti-cancer agents acquire resistance over the course of therapy and that not all cancers respond to treatment with taxane therapy. The inventors have demonstrated that composition comprising a combretastatin and a taxane and/or a platinum coordination compound is surprisingly effective in treating tumors that are refractive either to a combretastatin or to one or more anticancer agents.

Refractive tumors or cancers can be identified as those tumors from patients who have initially failed to respond to treatment with an anti-cancer agent or who have developed resistance during the course of treatment. Further, certain cancers are known to be intrinsically resistant or develop resistance to treatment with a particular anti-cancer agent. For example, colorectal cancers or melanomas are known to be innately resistant to taxane therapy and ovarian and lung cancers (e.g. small and non-small lung cancer) and are prone to acquired taxol resistance (Monzo et al., Proc. A.A.C.R., 38: 251 (#1689), (1997); Giannakakou et al., J. Biol. Chem. 272, 17118-17125, (1997); Ohta et al., Jpn. J. Cancer Res. 85, 290-297, (1994); Kavallaris et al., J. Clin, Invest. 100:1282-1293, (1997)). Other exemplary refractive tumors include those that are commonly resistant to cisplatin or carboplatin such as cervical cancer, ovarian cancer, fallopian tube cancer, or primary carcinoma of the peritoneum.

Alternatively, patients with acquired or intrinsic drug resistance can be identified by obtaining tumor tissue sample and conducting sequence or expression analysis of genes associated with drug resistance using techniques that are well-known in the art. Examples of genes that are generally associated with drug resistance include the multi-drug resistance genes (e.g. MDR1 ), P-glycoprotein, annexin I, interleukin 6 (IL-6), interleukin 8 (IL-8), macrophage inflammatory protein 2α (MIP2α), natural killer cell enhancing factor B (NKEFB). Methods for analysis of these genes are described for e.g. in U.S. Pat. No. 6,737,240. Other genes are specifically associated with a resistance to a particular anti-cancer agent. For example, taxol-resistance genes include the β-tubulin gene and the Taxol Resistance Associated Gene-3 (“TRAG-3”) which are overexpressed in certain patients (see for e.g. U.S. Pat. No. 6,362,321, U.S. Pat. No. 6,251,682, Wahl et al., Nature Medicine, 2:72-79, (1996); Horwitz et al., Natl. Cancer Inst. 15:55-61, (1993); Haber et al., J. Biol. Chem. 270:31269-31275, (1995); and Giannakakou et al., J. Biol. Chem. 272:17118-17125, (1997)).

Certain genes associated with resistance to cisplatin or carboplatin include the glutathione-S-transferase (GST) and metallothionein genes, the oncogenes H-ras (Sklar, et al., Cancer Res. 48: 793-797 (1988); Isonishi et al., Cancer Res. 51: 5903-5909 (1991); Peters et al., Int. J. Cancer 54: 450-455 (1993)), myc (Niimi et al., Br. J. Cancer 63: 237-241, (1991)), trk (Peters (1993), ibid.) and fos (Scanlon et al., Proc. Natl. Acad. Sci. USA, 88: 10591-10595, (1991)) and the DNA repair genes ERCC-1 (Reed et al., Proc. A.A.C.R., 30: 488 (1989)) and XPAC. Methods for assaying tumors for resistance to platinum coordinating compounds are known in the art (see, for example, U.S. Pat. Nos. 5,434,046; 5,703,336; 5,846,725; 5,942,389; and 6,046,044).

Combretastatin resistant tumors include those with a rim of peripherally-oxygenated tumor cells that remain viable following combretastatin-induced blood flow shutdown and tumor hypoxia. These tumors can be identified by standard imaging techniques known in the art, including, without limitation, magnetic resonance imaging (MRI), positron-emission tomography (PET), computerized fluorescent tomography (CRT), fluorescence-based imaging, or scintographic imaging of hypoxia-sensitive markers (see for example Sengupta et al., Faseb J., 2004, Stevenson et al., J. Clin. Oncol., 21(23):4428-38 (2003); Galbraith et al., J Clin Oncol. 21(15):2831-42 (2003); Anderson et al., J Clin Oncol., 21(15):2823-30 (2003), Siim et al., Cancer Res. 60(16):4582-8 (2000), and Maxwell et al., Int. J. Radiat. Oncol. Biol. Phys. 42(4):891-4, (1998)).

e) Sequence of Administration

Without wishing to be bound by any theory of action, certain anticancer agents may be most efficacious at relatively high tumor concentrations, but are rapidly cleared from tumor tissue. For such agents, the present inventors have found that simultaneous administration of a combretastatin and the at least one other anticancer agent can potentiate the effect of the combination. Simultaneous administration allows the other anticancer agent to rapidly accumulate to a peak concentration in tumor tissue, yet “traps” the agent as the vasculature clearing tumor tissue is disrupted by the combretastatin compound. Such agents are termed herein “Peak Tumor Concentration Agents”. Peak Tumor Concentration Agents may be administered simultaneously with, or within close temporal proximity to, the combretastatin compound.

Other agents, for example, need not be present at high concentrations, but are most effective during a relatively short period of the overall cell cycle. As such agents can become protein-bound and inactive over time when remaining in contact with tumor tissue, they are therefore most efficacious under conditions where a continuing supply of the agent reaches the tumor. Potentiation of the efficacy of combination therapy in these cases can be obtained by administering the anticancer agent and combretastatin compound sequentially, with sufficient delay between administrations to allow the action of one of the agents before the other. Thus, when such anticancer agent is administered first, followed by a delay before administering the combretastatin, the anticancer agent may reach the tumor tissue over a sufficient duration to allow action of the compound, with subsequent administration of the combretastatin compound further damaging tumor tissue.

When the combretastatin compound is administered first, followed by a delay to allow blood flow to the tumor to resume before administering the anticancer agent, the tumor is initially weakened by the combretastatin compound, followed by further damage to the tumor by the anticancer agent. In this latter case, duration of anticancer agent tumor concentration is more significant than peak concentration. The damage to tumor vasculature by the initial administration of the combretastatin compound does not prevent the relatively low concentration of anticancer agent needed from reaching the tumor tissue once blood flow resumes. Such agents are termed herein “Duration Exposure Agents”. Duration Exposure Agents and the combretastatin compound may thus be administered sequentially, with either administration of the combretastatin compound first, followed by the anticancer agent, or vice versa, provided that a sufficient delay is allowed between administrations to potentiate the combination. In yet an additional embodiment of the methods of the invention, certain agents are most efficacious when present at relatively high concentrations in tumor tissue over a longer duration (i.e., maximizing the “area under the curve” (AUC) of a plot of concentration over time). Administering such agents first, followed by a delay before administering the combretastatin compound, allows action of the anticancer agent, with subsequent administration of the combretastatin compound further weakening the tumor tissue. For such agents, administration of the anticancer agent first avoids premature damage to tumor vasculature and allows sufficient concentrations of anticancer agent to reach the tumor. Such agents are termed herein “High AUC Agents”. High AUC Agents and the combretastatin A-4 compound or combretastatin A-1 compound may thus be administered sequentially, with administration of the High AUC Agent preceding administration of the combretastatin compound, provided that a sufficient delay is allowed between administrations to potentiate the combination.

Such agents can preferably be administered, for example, within 24 hours of the administration of the combretastatin compound, such as within 2-24 hours prior, 3-24 hours prior, 6-24 hours prior, 8-24 hours prior, or 12 to 24 hours prior to administration.

Whether administered simultaneously or sequentially, the combretastatin compound and the at least one anticancer agent can be administered in any amount or by any route of administration effective for the modulation of tumor growth or metastasis, especially treatment of cancer as described herein.

f) Methods for Selecting Patients and Prognosing Treatment

In another aspect, the invention provides methods for selecting patients for treatment with the anti-cancer agents disclosed herein, in particular a combretastatin compound, as well as methods for prognosing the response of the patient to the treatment, and methods for monitoring the course of treatment with the anticancer agent.

The methods include determining the level of a biomarker in a biological sample derived from a patient previously treated with the anti-cancer agent. The methods of the invention employ granulocyte levels, in particular neutrophil levels, as a biomarker. Granulocytes (also referred to as polymorphonuclear granulocytes or “PMNs”) comprise 60-70% of normal blood leukocytes and are also found in extravascular sites. Granulocytes (e.g. neutrophils, basophils, or eosinophils) have a cytotoxic and/or cytolytic function and can phagocytose or lyse tumor cells. The inventors have discovered that granulocyte levels (e.g. neutrophil levels) substantially increase in patients following treatment with an anti-cancer agent (e.g. a combretastatin), and that when correlated with tumor response, such a biomarker may be employed as a surrogate marker of clinical efficacy.

Granulocyte levels may be determined by any acceptable method that is known in the art. In one embodiment, neutrophil levels may be measured directly by measuring (e.g. counting) the number or density of granulocyte cells in a biological sample obtained from a patient treated with an anti-cancer agent. Methods for measuring the number of density of granulocytes include flow cytometry and differential cell staining. The value for the number or density of granulocyte cells may be an absolute or relative value (e.g. a neutrophil:lymphocyte ratio). In another embodiment, neutrophil cells are measured indirectly by counting the number of leukocytes (i.e. white blood cells) and subtracting the number of lymphocytes (e.g. T and B cells) in the sample, thereby obtaining the number of neutrophils in the sample.

In another embodiment, the granulocyte levels may be obtained by measuring the amount of a granulocyte-specific marker in a biological sample. Granulocyte-specific markers include gene products (i.e. gene transcripts (e.g. mRNA) or proteins) that are expressed by granulocytes and which are expressed at lower levels (or not at all) by non-granulocyte cells. Exemplary granulocyte-specific markers include chloroacetate esterase, Gr-1, neutrophil-specific antigen, the gelatinase and lactoferrin granule proteins, and calprotectin (a neutrophil-specific marker). Levels of a granulocyte-specific gene product may be measured with a probe. Suitable probes include, for example, cDNA, riboprobes, and antibodies. The type of probe used will generally be dictated by the particular situation, such as riboprobes for in situ RNA hybridization, cDNA for Northern blotting, and antibodies for Western Blotting or ELISA. The most preferred probes are those directed to nucleotide or polypeptide regions that are unique to the neutrophil-specific gene product. The form of labeling of the probes may be any that is appropriate, such as the use of radioisotopes. Labeling with radioisotopes may be achieved, whether the probe is synthesized chemically or biologically, by the use of suitably labeled bases. Other forms of labeling may include enzyme or antibody labeling such as is characteristic of ELISA.

A method for selecting a patient for further treatment with an anticancer agent (e.g. a combretastatin) may be based on the level of granulocyte biomarker observed in a biological sample obtained from the patient. The method comprises treating the patient with a first dose of anti-cancer agent, obtaining a biological sample from the patient, and measuring the level of granulocytes in the biological sample, and selecting the patient for treatment based at least in part on the level obtained. The patient may be selected for continued treatment with the anticancer agent if increased granulocyte levels are observed following initial treatment with the anticancer agent. Alternatively, if granulocyte levels decrease or remain constant following treatment, the patient may be advised to discontinue treatment with the anticancer agent.

In another aspect, granulocyte levels may be used to monitor the progression of cancer in the patient following treatment with an anti-cancer agent (e.g. a combretastatin). The methods include determining the granulocyte level in the patient at a first time following treatment with the anti-cancer agent, determining the granulocyte level in the patient at a subsequent time following a treatment with the anticancer agent. For example, the first measurement may be performed at a time just following a first dose of anti-cancer agent, while a second measurement may be performed at a second time that follows the first dose or a second or subsequent dose of anti-cancer agent. Granulocyte levels obtained at said first time and second times may then be compared. Decreased levels of granulocytes at the second time relative to the first time may be used to support a diagnosis that the tumor has progressed or relapsed (i.e. continued growth of the tumor). Increased levels of granulocytes at the second time relative to the first time may be used to support a diagnosis that the tumor has regressed (i.e. tumor shrinkage).

In another aspect, the invention provides a method of assessing, predicting or prognosing the likelihood of a patient's response (e.g. tumor regression or remission) to treatment with an anti-cancer agent, and in particular a combretastatin. Efficacy of anti-cancer agents can be predicted and the probable clinical course of a patient suffering from cancer can be determined by measuring granulocyte levels in a biological sample obtained from the patient. For example, an increase in granulocyte levels correlates with the increased likelihood of a tumor response. The presence of elevated granulocyte levels in the biological sample of the patient is indicative of a response of the tumor to treatment with the anti-cancer agent (e.g. a combretastatin). Conversely, lower levels than certain baselines can also be used to indicate the lack of response of the tumor to treatment. For example, an elevation of neutrophil levels (i.e. neutrophilia) is determined by comparing the post-treatment neutrophil levels with a baseline level (e.g. the same or different patient than prior to treatment with the anticancer agent). Such a patient is predicted to have a favorable prognosis.

Increases or decreases in relative or absolute granulocyte levels of more than 1.0% from baseline may be used to make any of the determinations described above. Preferably, the increase or decrease in granulocyte levels, is greater than 2.0%, 3.0%, 4.0%, 5.0%, 7.0%, 10%, 15%, 20%, 25%, 30%, 40%, 50%, or more. While the exact baseline level is somewhat arbitrary (as the numerical cut off value may be shifted upward or downward with an attendant loss of accuracy in the prognostic utility of the test), it is well within the skill of one of ordinary skill in the art to determine the appropriate baseline level, by either using the experimental methods disclosed herein, for example, establishing granulocyte levels in patients that have not been administered the anti-cancer agent that is evaluated. Further, as will be appreciated by those of ordinary skill in the art, the evaluation of the treatment may also be based upon an evaluation of the symptoms or clinical end-points of the associated disease.

The comparison of a subject's granulocyte levels employs measurements obtained from biological samples collected from the subject at different sample times. A first biological sample, if necessary, may be obtained at any time prior to treatment with an anti-cancer agent. A second biological sample is preferably obtained within 24 hours of treatment with the anticancer agent (e.g. a combretastatin). In more preferred embodiment, a second biological sample is obtained less than 6 hours following the administration of an anticancer agent (e.g. a combretastatin). The preferred time to obtain the second biological sample from the subject is at 4 hours following the administration of the anticancer agent.

Biological samples, which can be screened for granulocyte levels, are samples containing granulocytes, preferably neutrophils. Examples include, but are not limited to, tumor biopsy samples and blood or serum samples obtained from the patient. In a preferred embodiment, the biological sample is obtained from the blood of the patient.

g) Pharmaceutical Compositions

As explained above, the present methods can, for example, be carried out using a single pharmaceutical composition comprising both a combretastatin compound and one or more anticancer agent(s) when administration is to be simultaneous or using two or more pharmaceutical compositions separately comprising a combretastatin compound and anticancer agent(s) when administration is to be simultaneous or sequential. Such pharmaceutical compositions can comprise, inter alia, at least one anticancer agent and/or a combretastatin compound, such as a CA4P compound or CA1P compound and a pharmaceutically acceptable carrier. The phrase “pharmaceutically acceptable” refers to molecular entities and compositions that are physiologically tolerable and preferably do not produce an allergic or similar untoward reaction, such as gastric upset, dizziness and the like, when administered to a human.

Preferably, as used herein, the term “pharmaceutically acceptable” means approved by a regulatory agency of the Federal or a state government or listed in the U.S. Pharmacopoeia or other generally recognized pharmacopeias for use in animals, and more particularly in humans. The term “carrier” refers, for example to a diluent, adjuvant, excipient, auxiliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. E. W. Martin describes suitable pharmaceutical carriers in “Remington's Pharmaceutical Sciences”.

A pharmaceutical composition of the present invention can be administered by any suitable route, for example, by injection, by oral, pulmonary, nasal or other forms of administration. In general, pharmaceutical compositions contemplated to be within the scope of the invention, comprise, inter alia, pharmaceutically acceptable diluents, preservatives, solubilizers, emulsifiers, adjuvants and/or carriers. Such compositions can include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, Pa. 18042) pages 1435-1712 which are herein incorporated by reference. A pharmaceutical composition of the present invention can be prepared, for example, in liquid form, or can be in dried powder, such as lyophilized form. Particular methods of administering such compositions are described infra.

h) Methods of Administration

As explained above, the present invention is directed towards methods for modulating tumor growth and metastasis comprising, inter alia, the administration of a combretastatin compound, such as a CA4P compound or a CA1P compound, and at least one anticancer agent. The agents of the invention can be administered separately (e.g., formulated and administered separately), or in combination as a pharmaceutical composition of the present invention. Administration can be achieved by any suitable route, such as parenterally, transmucosally, e.g., orally, nasally, or rectally, or transdermally. Preferably, administration is parenteral, e.g., via intravenous injection. Alternative means of administration also include, but are not limited to, intra-arteriole, intramuscular, intradermal, subcutaneous, intraperitoneal, intraventricular, and intracranial administration/or by injection into the tumor(s) being treated or into tissues surrounding the tumor(s).

The combretastatin compound, such as a CA4P compound or CA1P compound and anticancer agent may be employed in any suitable pharmaceutical formulation, as described above, including in a vesicle, such as a liposome [see Langer, Science 249:1527-1533 (1990); Treat et al., in Liposomes in the Therapy of Infectious Disease and Cancer, Lopez-Berestein and Fidler (eds.), Liss: New York, pp. 317-327, see generally, ibid] Preferably, administration of liposomes containing the agents of the invention is parenteral, e.g., via intravenous injection, but also may include, without limitation, intra-arteriole, intramuscular, intradermal, subcutaneous, intraperitoneal, intraventricular, and intracranial administration, or by injection into the tumor(s) being treated or into tissues surrounding the tumor(s).

In yet another embodiment, a pharmaceutical composition of the present invention can be delivered in a controlled release system, such as using an intravenous infusion, an implantable osmotic pump, a transdermal patch, liposomes, or other modes of administration. In a particular embodiment, a pump may be used [see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)]. In another embodiment, polymeric materials can be used [see Medical Applications of Controlled Release, Langer and Wise (eds.)/CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)]. In yet another embodiment, a controlled release system can be placed in proximity of the target tissues of the animal, thus requiring only a fraction of the systemic dose [see, e.g., Goodson, in Medical Applications of Controlled Release, supra, vol. 2, pp. 115-138 (1984).]. In particular, a controlled release device can be introduced into an animal in proximity of the site of inappropriate immune activation or a tumor. Other controlled release systems are discussed in the review by Langer [Science 249:1527-1533 (1990)].

The following examples are provided to illustrate embodiments of the invention. They are not intended to limit the invention in any way.

### EXAMPLES

The following protocols are provided to facilitate the practice of Examples I and II.
Drug administration: For administration to rodents, CA4P was dissolved in normal saline (0.9% NaCl). Paclitaxel was dissolved in a 50/50 mixture of ethanol and Cremophor® and stored at 4° C.; final dilution of paclitaxel was obtained immediately before drug administration with NaCl 0.9%. Fresh preparation of paclitaxel was employed to avoid precipitation. CPT-11 was dissolved in normal saline.
The volume of all compounds injected was 0.01 ml/g of mice, and 0.005 ml/g of rats.
In Vivo Antitumor Testing: The following tumor models were used: A2780 human ovarian carcinoma, the murine fibrosarcoma M5076 and M5076/ddp (resistant to cisplatin and carboplatin).

The human tumors were maintained in Balb/c nu/nu nude mice. M5076 and M5076ddp were maintained in C57BL/6 mice. Tumors were propagated as subcutaneous transplants in the appropriate mouse strain using tumor fragments obtained from donor mice.

The following tumors were passaged in the indicated host strain of mouse: murine M5076 fibrosarcoma (M5076) in C57B1/6 mice; human A2780 ovarian carcinomas in nude mice. Tumor passage occurred biweekly for murine tumors and approximately every two to three weeks for the human tumor line. With regard to efficacy testing, M5076 and M507 6ddp tumors were implanted in (C57B1/6×DBA/2)F1 hybrid mice, and human tumors were implanted in nude mice. All tumor implants for efficacy testing were subcutaneous (sc).

The required numbers of animals needed to detect a meaningful response were pooled at the start of the experiment and each was given a subcutaneous implant of a tumor fragment (˜50 mg) with a 13-gauge trocar. For treatment of early-stage tumors, the animals were again pooled before distribution to the various treatment and control groups. For treatment of animals with advanced-stage disease, tumors were allowed to grow to the predetermined size window (tumors outside the range were excluded) and animals were evenly distributed to various treatment and control groups. Treatment of each animal was based on individual body weight. Treated animals were checked daily for treatment related toxicity/mortality. Each group of animals was weighed before the initiation of treatment (Wt1) and then again following the last treatment dose (Wt2). The difference in body weight (Wt2−Wt1) provides a measure of treatment-related toxicity.

Tumor response was determined by measurement of tumors with a caliper twice a week, until the tumors reach a predetermined “target” size of 1 gm. Tumor weights (mg) were estimated from the formula:
Tumor weight=(length×width2)÷2
Antitumor activity was evaluated at the maximum tolerated dose (MTD) which is defined as the dose level immediately below which excessive toxicity (i.e. more than one death) occurred. The MTD was frequently equivalent to OD. When death occurs, the day of death was recorded. Treated mice dying prior to having their tumors reach target size were considered to have died from drug toxicity. No control mice died bearing tumors less than target size. Treatment groups with more than one death caused by drug toxicity were considered to have had excessively toxic treatments and their data were not included in the evaluation of a compound's antitumor efficacy.

Tumor response end-point was expressed in terms of tumor growth delay (T−C value), defined as the difference in time (days) required for the treated tumors (T) to reach a predetermined target size compared to those of the control group (C).

To estimate tumor cell kill, the tumor volume doubling time was first calculated with the formula:
TVDT=Median time (days) for control tumors to reach target size−Median time (days) for control tumors to reach half the target size and, Log cell kill=T−C÷(3.32×TVDT).

Statistical evaluations of data were performed using Gehan's generalized Wilcoxon test.

### Example 1

### Two-Component Combination Chemotherapy

M507 6DDP is a murine fibrosarcoma that has developed resistance to cisplatin and cross-resistance to carboplatin. The in vivo antitumor activity of CA4P or CA1P in combination with each of these platinum coordination compounds and CPT-11 were evaluated in M5076DDP tumor bearing mice.

i) CA4P+Cisplatin

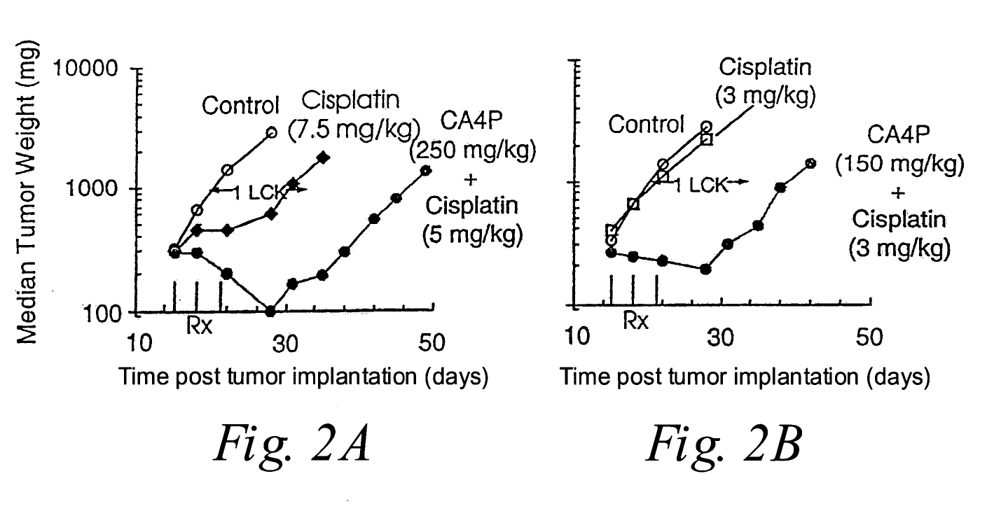

CA4P treatment of mice bearing advanced (300 mg) M507 6DDP tumors using an everyday×10 (Monday thru Friday) schedule produced moderate but significant antitumor effects. At its optimal dose (150 mg/kg/inj), combretastatin A-4 phosphate disodium salt yielded 1.1 log cell kill (LCK). In comparison, single agent cisplatin produced 0.8 log cell kill (LCK) at its maximum tolerated dose (MTD) of 7.5 mg/kg when injected every 4 days for 3 doses (Q4D×3). In comparison, the combination of CA4P (250 mg/kg/inj)+Cisplatin (5 mg/kg/inj), administered simultaneously, achieved a 2.0 LCK, which is consistent with a synergistic effect between CA4P and cisplatin (FIG. 2A).

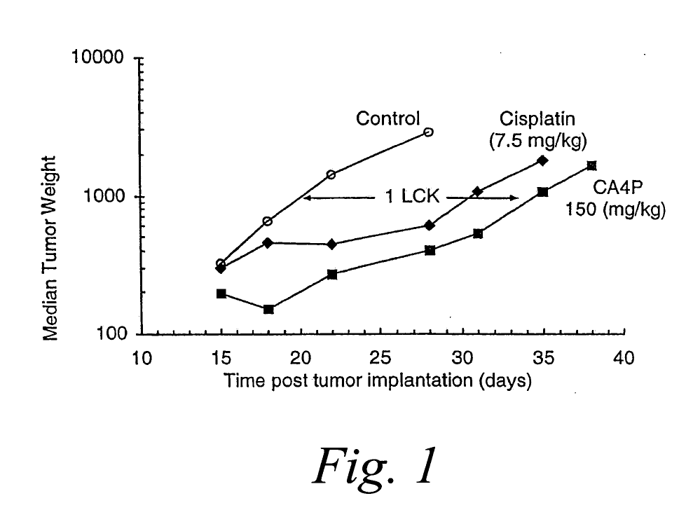

It is of interest that the combination produced significant shrinkage of tumors following treatment, whereas single agent cisplatin did not (FIG. 1). Another noteworthy aspect of this synergistic combination regimen is the ability of CA4P to substantially improve the efficacy of an otherwise inactive (lower) dose of cisplatin (FIG. 2B).

ii) CA4P+Carboplatin:

CA4P also produced synergistic antitumor activity against large sc M5076 tumors (H300 mg) when used in combination with carboplatin. In this sensitive tumor model, carboplatin alone produced 1.4 LCK, but with no tumor regression, at its MTD of 90 mg/kg/inj, iv, Q4D×3. A separate experiment showed that single agent CA4P administered Q4D×3 had no activity in this model.

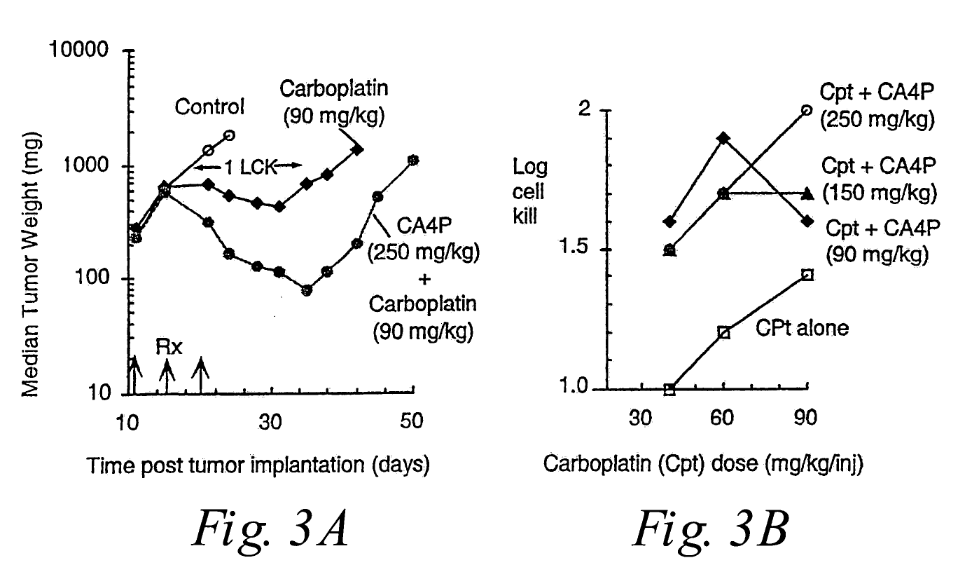

In comparison the best combination yielded 2.0 LCK, which was accompanied by significant tumor shrinkage (FIG. 3A). These data are consistent with synergistic anti-tumor activity between CA4P and Carboplatin.

Two important aspects of the tumor response elicited by the CA4P+carboplatin combination regimen' are: (1) the optimal CA4P dose required for therapeutic synergy (<90 mg/kg/inj) was significantly lower than its MTD as a single agent (>250 mg/kg/inj) (FIG. 3B); (2) the carboplatin dose (90 mg/kg/inj when administered as single agent) required to produce optimal antitumor effects, is greatly reduced when used in combination with CA4P (FIG. 3B).

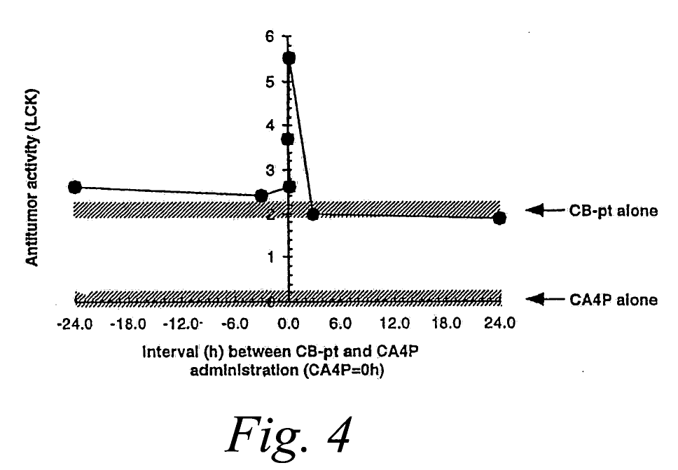

Sequence timing studies indicated that Carboplatin (“CB-pt”) and CA4P are preferably administered more or less simultaneously (FIG. 4). Most preferably carboplatin is administered immediately before CA4P. The tumor model shown in this graph is M5076ddp (a platinum resistant variant of M5076 murine fibrosarcoma).

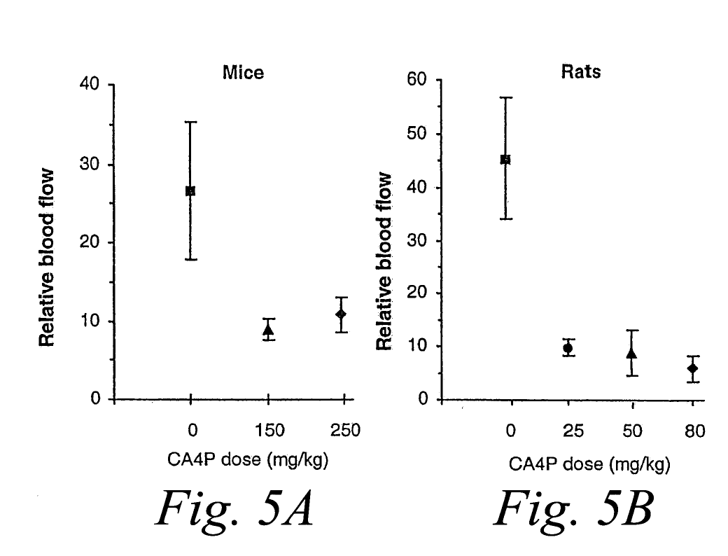

The effects of CA4P disodium salt on tumor perfusion were also studied using the Evans blue dye uptake assay. Mice or rats bearing sc A2780 human ovarian carcinoma were administered an iv dose of CA4P disodium salt. An hour later, Evans blue was injected iv. The amount of Evans blue accumulated in the tumor is proportional to the blood flow through the tumor. Using this technique, it was shown that CA4P dramatically inhibited blood flow to the tumors, both in mice and rats, causing at optimal dose a 67% and 87% reduction of tumor blood flow, respectively (FIG. 5A and 5B).

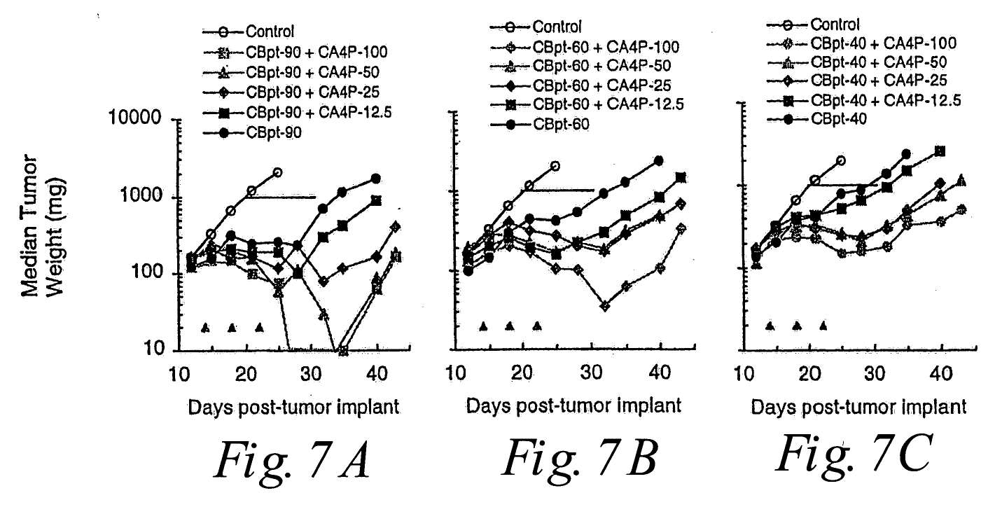

Due to the robust therapeutic synergism with cisplatin and carboplatin as shown herein, the doses of CA4P disodium salt were lowered as human pharmacokinetics data indicate that preferred CA4P dosing is considerably lower (free acid 45-63 mg/m²). A study was therefore conducted to determine the minimum CA4P dose needed for combination therapy with carboplatin in the modestly carboplatin resistant murine fibrosarcoma M507 6/DDP. Using doses and treatment regimen (iv, q4d×3) of CA4P that have no single agent activity, it was demonstrated that CA4P at doses as low as 12.5-25 mg/m² were sufficient to enhance the antitumor activity of carboplatin administered at a range of dose levels. See FIGS. 7A, 7B and 7C.

iii) CA4P+CPT-11

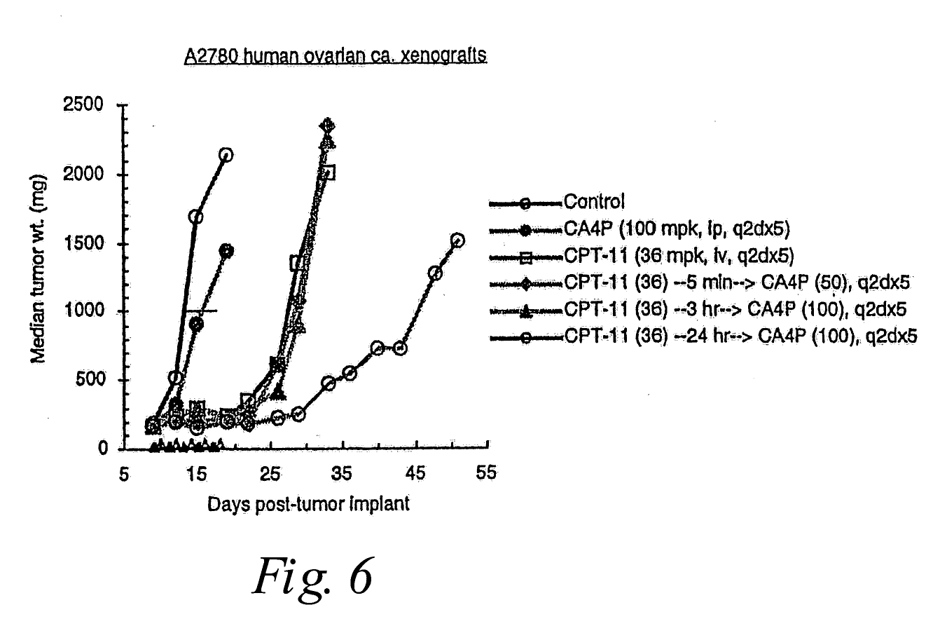

A combination chemotherapy study was conducted to evaluate the antitumor activity of combined CPT-11 and CA4P disodium salt treatment. Various dosing schedules were used in accordance with the invention ranging from administering the two agents virtually simultaneously (5 min apart) to CPT-11 preceding CA4P by 3 or 24 hrs. At its MTD, CPT-11 produced 3.3 LCK. Administering the two agents simultaneously or 3 hr apart gave equivalent results to CPT-11 alone. However, when CPT-11 preceded CA4P by 24 hr, an enhanced antitumor effect was observed (FIG. 6) demonstrating a preferred embodiment of the invention.

iv) CA4P+Paclitaxel

The present invention contemplates, for example, the administration of a combretastatin compound, such as CA4P, with paclitaxel or with paclitaxel and carboplatin. A number of studies were conducted to determine an optimal treatment schedule, i.e., the sequence or the order, in which the two agents, CA4P and paclitaxel are administered. This consideration is deemed particularly important for this combination for two reasons: 1) CA4P is a tubulin depolymerizer while paclitaxel is a tubulin polymerizer, thus there may be potential for interaction at the tubulin level; and 2) CA4P inhibits tumor blood flow which may affect the regional, micro-pharmacokinetics of paclitaxel in the tumors as well as the tumoral proliferative state.

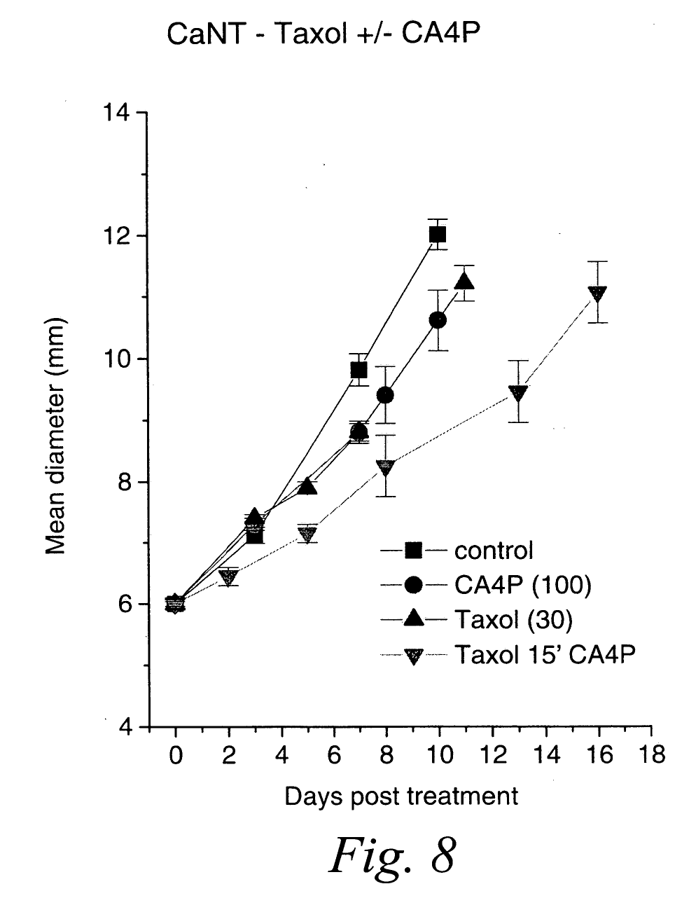

An initial study was conducted to assess the effects of administering paclitaxel (30 mg/kg) together with CA4P (100 mg/kg). An interval of 15 min between the administrations of the two agents was employed. Results indicate that administration of the two agents as combination therapy was surprisingly synergistic to overall efficacy of the combination in this model (FIG. 8). This result was particularly unexpected in view of the mechanisms of action that are commonly accepted for each agent. Taxol is known to exert its effects on tumor growth control by binding to polymerized microtubules of a rapidly proliferating tumor cell and stabilizing them, thereby inhibiting their de-polymerization and arresting tumor cell division (see, for example, Gelmon K., et al. The Lancet, (1994); 344: 1267-1272). In contrast, CA4P has been ascribed an opposing mechanism of action in which binding of the agent to β-tubulin monomers (as opposed to polymerized tubulin) prevents their assembly into microtubules (Sackett D., et al. Pharmacol Ther., (1993); 59(2):163-228).

v) CA1P+Carboplatin or Cisplatin

In order to better assess the therapeutic potential of CA1P, studies were conducted to evaluate three aspects of CA1P pharmacology: [1] antitumor efficacy as a single agent, [2] antitumor efficacy in combination with cisplatin, and [3] effects on tumor blood flow.

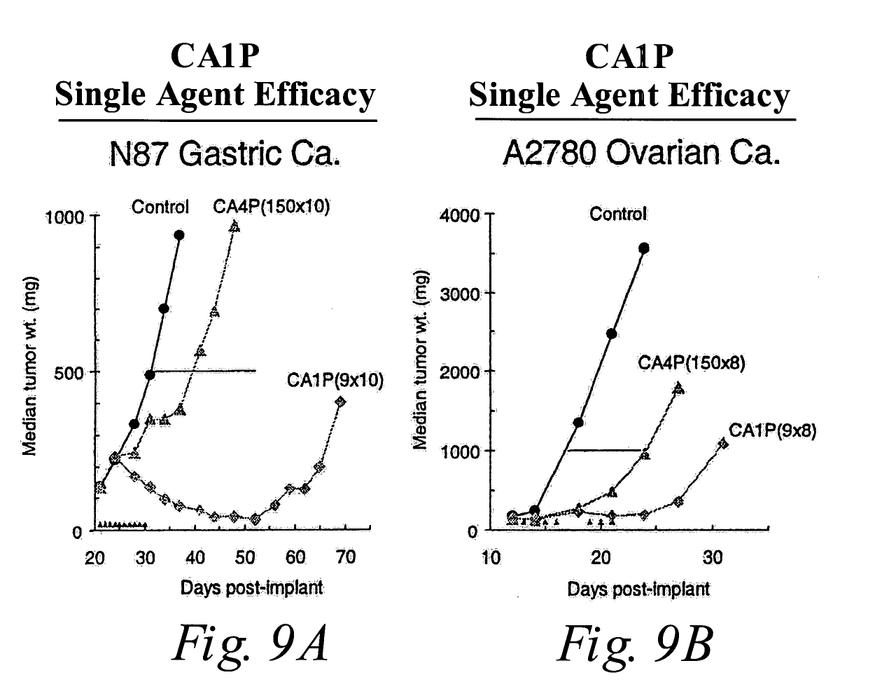

CA1P has demonstrated improved single agent activity in human tumor xenograft models, including N87 human gastric carcinoma, and the A2780 ovarian carcinoma. In A2780, CA1P achieved 2.1 LCK at its MTD of 9 mg/kg, ip, q1d×8, compared to 1.1 LCK for CA4P at 150 mg/kg, ip. See FIG. 9.

In tumor perfusion studies, CA1P sodium salt significantly inhibited tumor blood flow in both A2780 human ovarian tumor xenografts in mice and N87 gastric cancer tumor xenografts. When administered daily as a single agent for ten days to tumor bearing mice, CA1P sodium salt demonstrated equivalent blood flow inhibition to that observed with CA4P in human tumor xenografts in nude mice but was 5-10 times more potent.

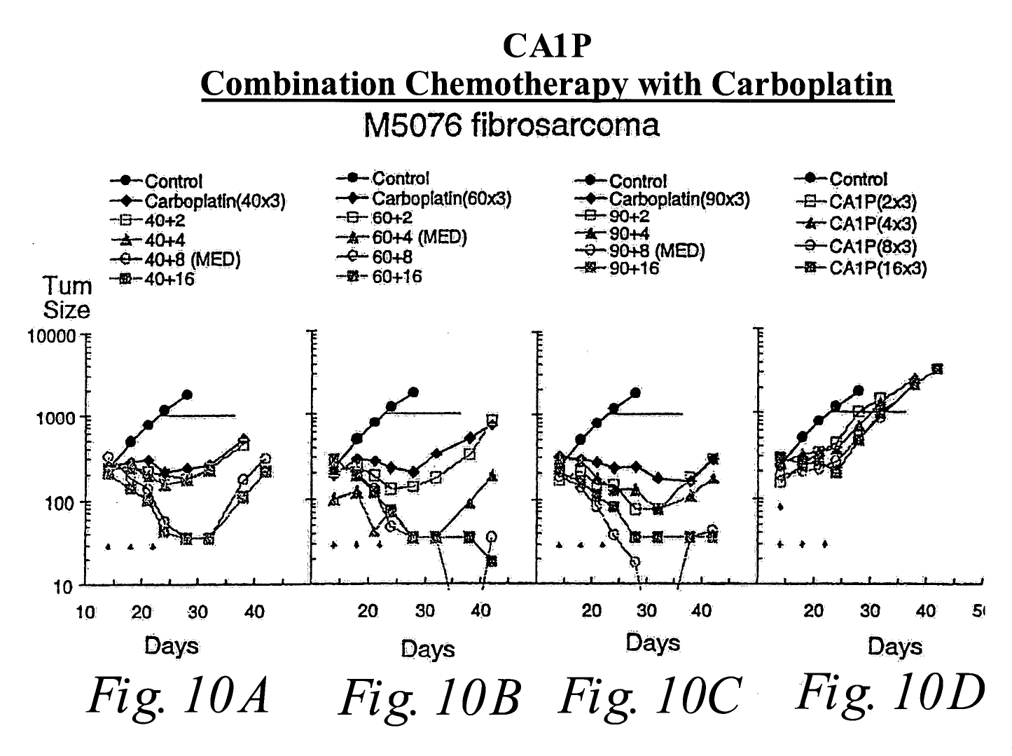

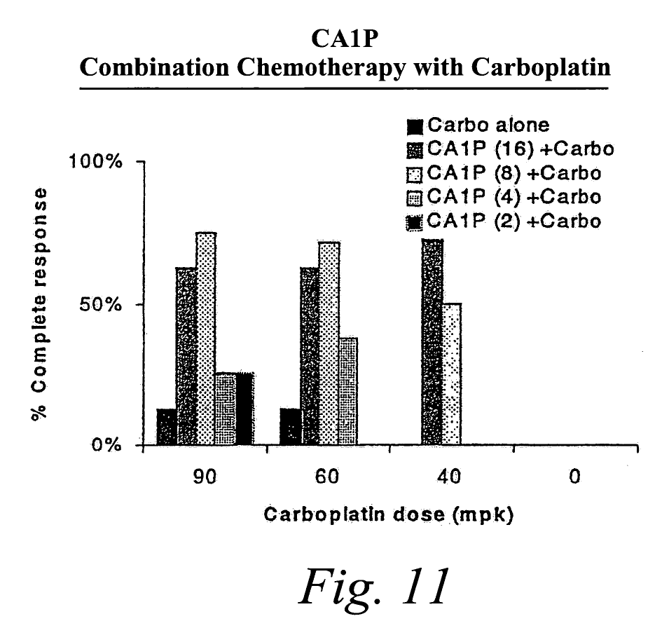

In a combination chemotherapy trial, therapeutic synergy was observed with carboplatin. As shown in FIG. 10, combination chemotherapy demonstrated that CA1P enhanced the antitumor activity of carboplatin in a manner similar to what had been observed for CA4P. Synergistic antitumor activity was also demonstrated. Advantageously, the minimum effective dose required for synergistic enhancement was considerably lower for CA1P (4-8 mg/kg) as compared to CA4P (25-50 mg/kg). Additionally, when CA1P is administered in combination with carboplatin, synergistic antitumor activity producing a complete response (disappearance of tumors) was observed. When either agent was administered alone, this response was not observed. See FIG. 11.

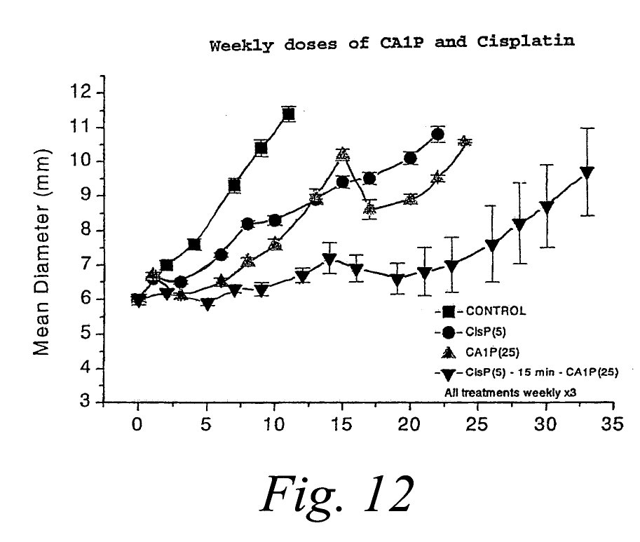

In additional studies, CA1P was administered in combination with cisplatin in a CaNT breast tumor model. As can be seen in FIG. 12, combined administration of cisplatin and CA1P acted synergistically to reduce tumor size.

### Example 2

### Three-Component Combination Chemotherapy

A study was conducted to assess the effects of administering a combretastatin in combination with both a taxane and a platinum coordination compound. The triple drug cocktail of CA4P in combination with the standard chemotherapeutic regimen of carboplatin and paclitaxel was chosen for investigation. In addition, a number of sequences of administration were studied.

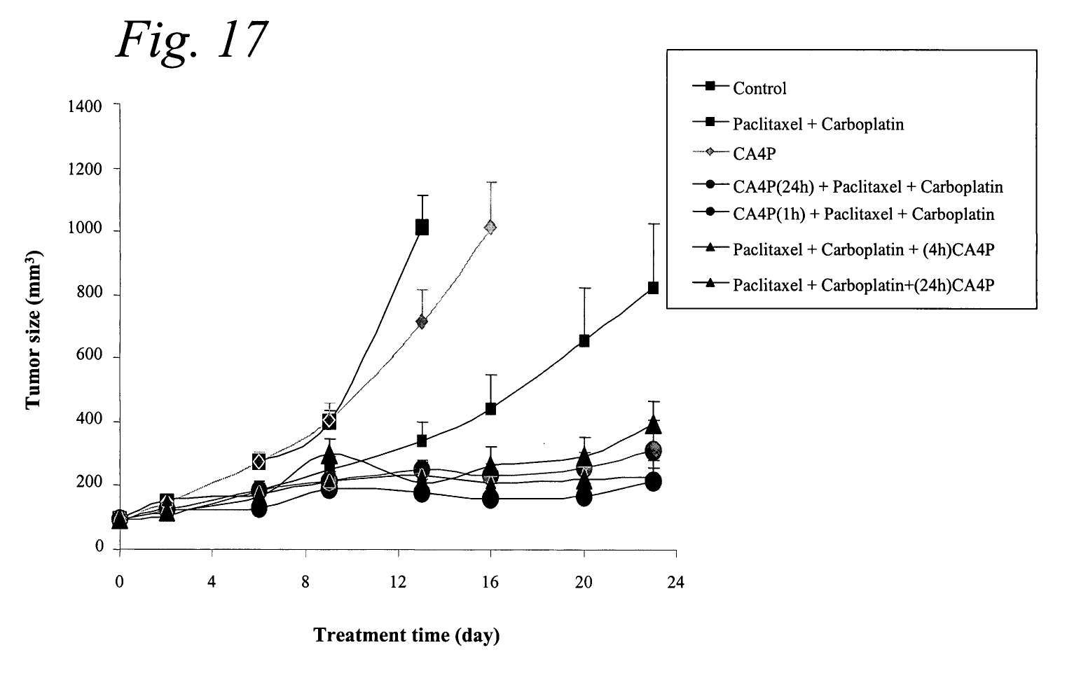

A human breast adenocarcinoma model was established by subcutaneous injection of cultured MDA-MB-231 cells in Fox Chase CB-17 SCID mice. When the average tumor size reached 50-100 mm³, mice were randomly divided into several groups (I-VII) with no significant difference in body weight and tumor size. On Day 0, each group was administered anti-cancer agents. Group I mice were administered saline carrier only as a control treatment. Group II mice received an intraperitoneal (i.p.) injection of paclitaxel at a dose of 9 mg/kg, followed 30 minutes later by an infusion of Carboplatin at a dose of 30 mg/kg. Group III mice receive CA4P disodium salt at a dose of 100 mg/kg. Group IV mice received the same dose of CA4P as Group III mice, followed 24 hours later by treatment with paclitaxel and carboplatin at the same doses as in Group II. Group V mice received CA4P as in Group IV, followed 1 hour later by treatment with paclitaxel and carboplatin. Group VI and VII mice received treatment with paclitaxel and carboplatin first. CA4P was administered either 4 hours (Group VI) or 24 hours later (Group VII). This treatment regime was continued once a week for 3 weeks. On Day 2, 6, 9, 13, 16, 20 and 23, tumors in each treatment group (n=2) were measured by width and length. Tumor size (i.e. volume) was calculated according to the following formula: Length×Width²×0.4. As is illustrated in FIG. 17, while the two component therapy of carboplatin and paclitaxel affected growth delay, the triple combination of CA4P, paclitaxel, and carboplatin almost completely inhibited tumor growth. Moreover, the administration of CA4P either simultaneously (within 1 hr) or sequentially (within 24 hours), in any order, led to synergistic anti-tumor effect.

### Example 3

### Treatment of Drug-Resistant Tumors

A study was conducted to assess the effects of administering a combretastatin in combination with both a taxane and a platinum coordination compound for treatment of drug resistant tumors. The effectiveness of a triple drug cocktail (a combretastatin+paclitaxel+carboplatin) was investigated in tumors that are resistant to a standard-line combination chemotherapy of carboplatin and paclitaxel.

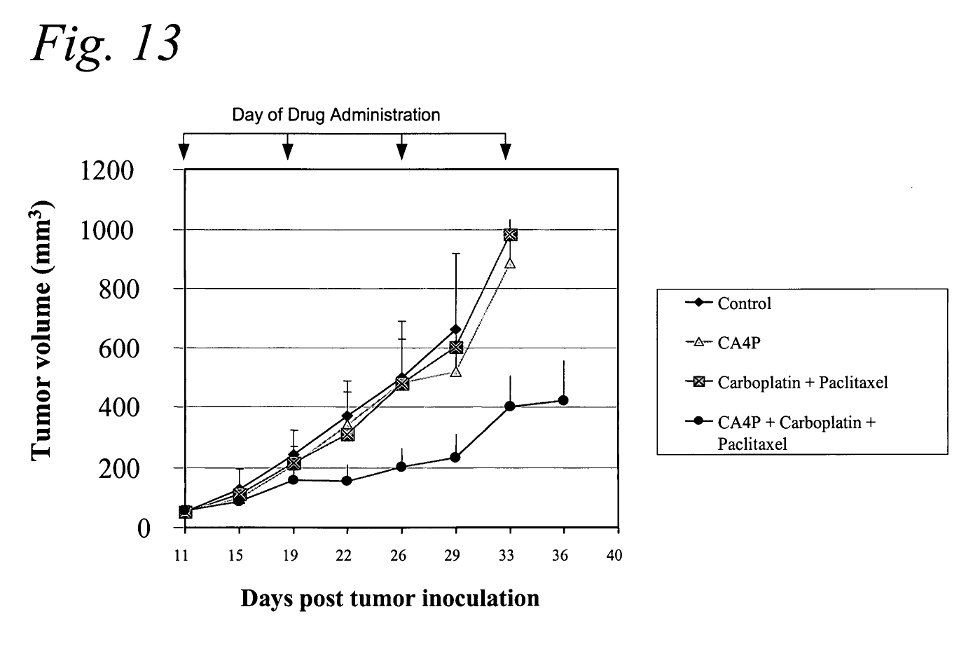

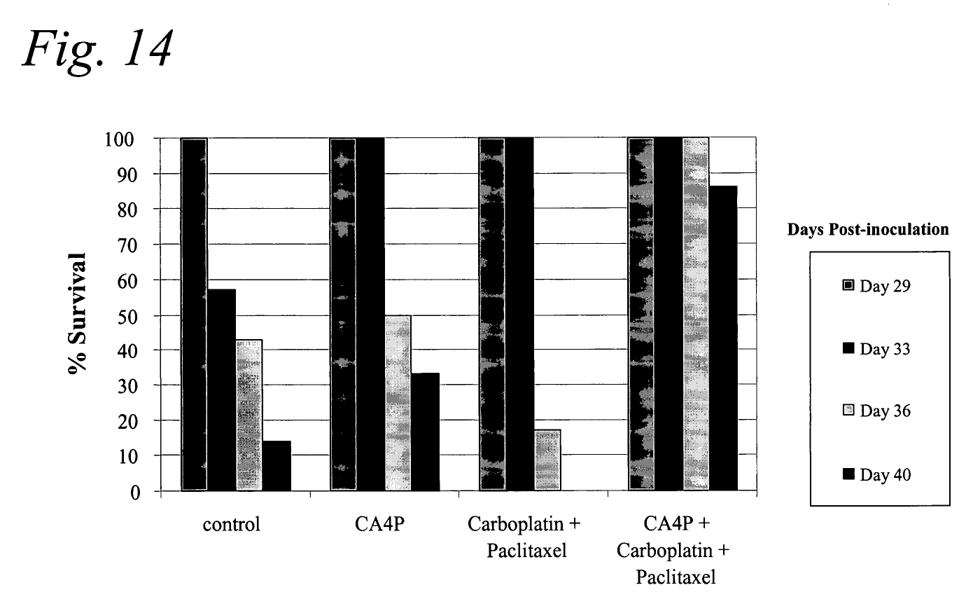

The multi-drug resistant ES2 human clear cell ovarian carcinoma was established by subcutaneous injection of cultured ES2 cells in Fox Chase CB-17 SCID mice. In one experiment, tumor-bearing mice were administered CA4P at a dose of 100 mg/kg (Group II), saline carrier only (Group I), an intraperitoneal (i.p.) injection of paclitaxel at a dose of 9 mg/kg, followed 30 minutes later by an infusion of carboplatin at a dose of 30 mg/kg (Group II), or CA4P (100 mg/kg) followed 24 hours later by paclitaxel and carboplatin as in Group II (Group IV). Treatment with anticancer agents was performed once a week for 4 weeks (i.e. on Day 11, 19, 26, and 33). Tumors in each treatment group (n=2) were measured every 4 days by width and length. Tumor size (i.e. volume) was calculated according to the following formula: Length×Width²×0.4. As is illustrated in FIG. 13, the two component therapy of paclitaxel and carboplatin alone was almost completely ineffective in controlling tumor growth. Moreover, CA4P alone was not significantly more effective than paclitaxel and carboplatin. However, the combination of CA4P, carboplatin and paclitaxel clearly reversed drug resistance and significant growth delay was achieved. Moreover, this triple combination clearly lengthened the survival of the tumor bearing animals (see FIG. 14). More than 80% of the animals treated with the triple combination were still alive at the conclusion of the experiment as compared to 30% with CA4P alone and none with paclitaxel and carboplatin.

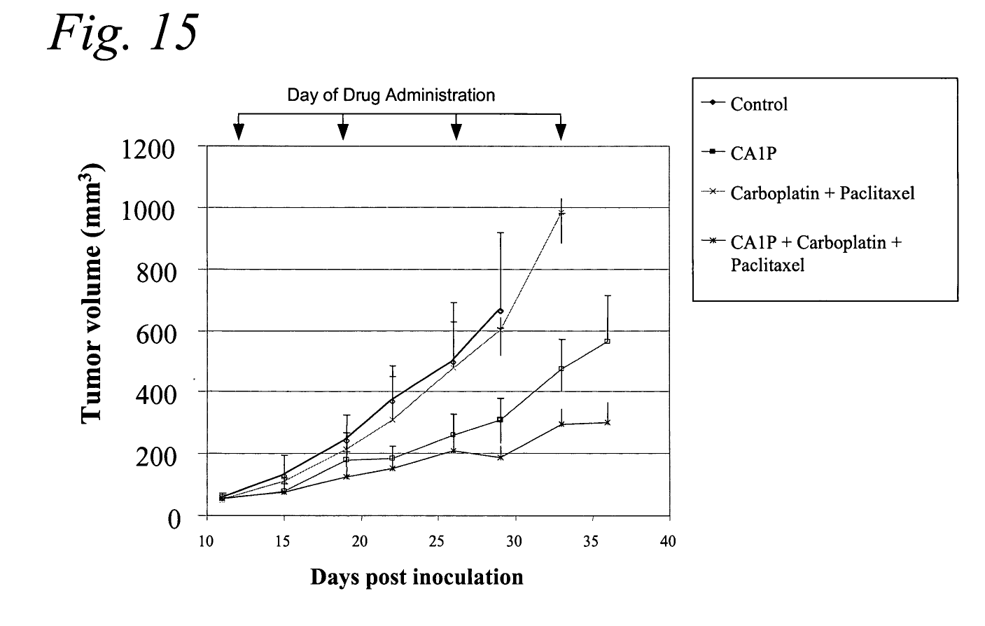

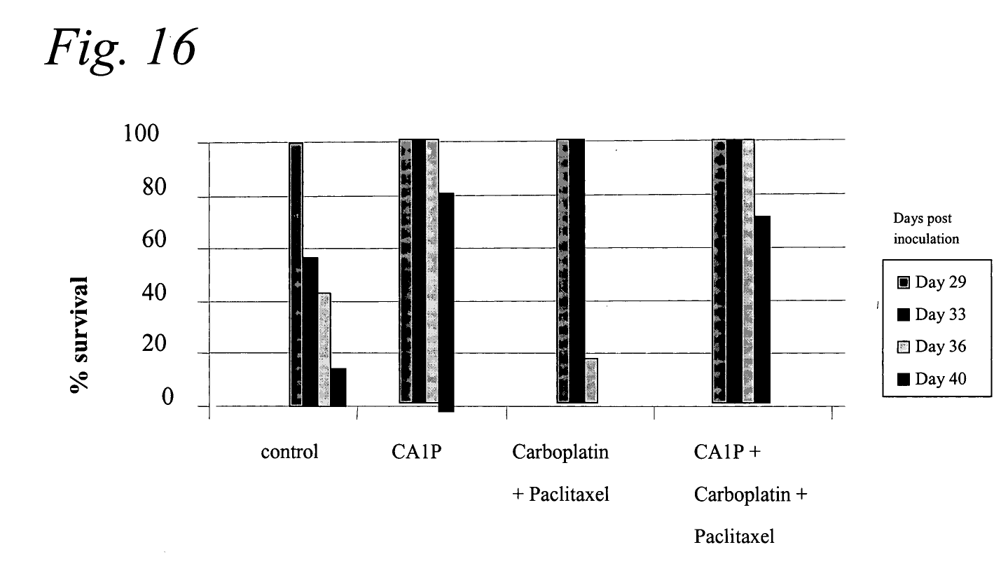

In a second experiment, tumor-bearing mice were administered CA1P and/or carboplatin and paclitaxel in the same treatment regime as the first experiment. As is illustrated in FIG. 15, the two component therapy of paclitaxel and carboplatin alone was again almost completely ineffective in controlling tumor growth. However, in contrast to CA4P, CA1P alone effected considerable anti-tumor growth delay, despite the multi-drug resistance phenotype. The combination of CA1P, carboplatin and paclitaxel further potentiated the reversal of drug resistance and synergistic effect on tumor growth delay was achieved. Moreover, this triple combination clearly potentiated the survivorship of the tumor bearing animals (see FIG. 16). More than 70% of the animals treated with the triple combination were still alive at the conclusion of the experiment while none of the animals treated with Paclitaxel and Carboplatin were still alive.

### Example 4

### Neutrophila as an Indicator of Clinical Prognosis

Hematological analysis was performed on whole blood obtained from patients participating in a Phase I trial of CA4P (see Rustin et al., J. Clin. Oncol. (2003)). All patients had histologically confirmed tumors and were either not amenable to standard curative therapy or were refractory to conventional therapy. Blood work was obtained from each patient 1 minute prior to treatment, every 15 minutes for the first hour post-treatment, and at 1.5, 2, 4, 8, 12 and 24 hours following infusion with the first dose of CA4P (52-114 mg/m²). Art-recognized hematology methods were used in the analysis.

All full or differential blood cell counts were performed using a hematology flow cytometer (Technicon H2, Bayer Inc.). Total white blood cell (“WBC” or leukocyte) and red blood cell (“RBC” or erythrocyte) counts were established using direct laser flow cytometry. A differential count for leukocyte cells of myeloid origin (e.g. eosinophils, neutrophils, monocytes) was obtained by fluorescent flow cytometry of cells stained for myeloperoxidase. Unstained cells were assumed to be lymphocytes and basophils. Lymphocyte counts were obtained by subtracting those cells that stained negative for a second, basophil-specific cell stain. Mean Corpuscular Volume (MCV) and Mean Corpuscular Haemoglobin (MCH) were also obtained by laser flow cytometry. The haematocrit (Hct) was calculated from the RBC count and the MCV. The coagulant status of each blood sample was determined by measuring fibrinogen content, prothrombin time, and kaolin partial thromboplastin time (when kaolin is used instead of micronised silica as the activator).

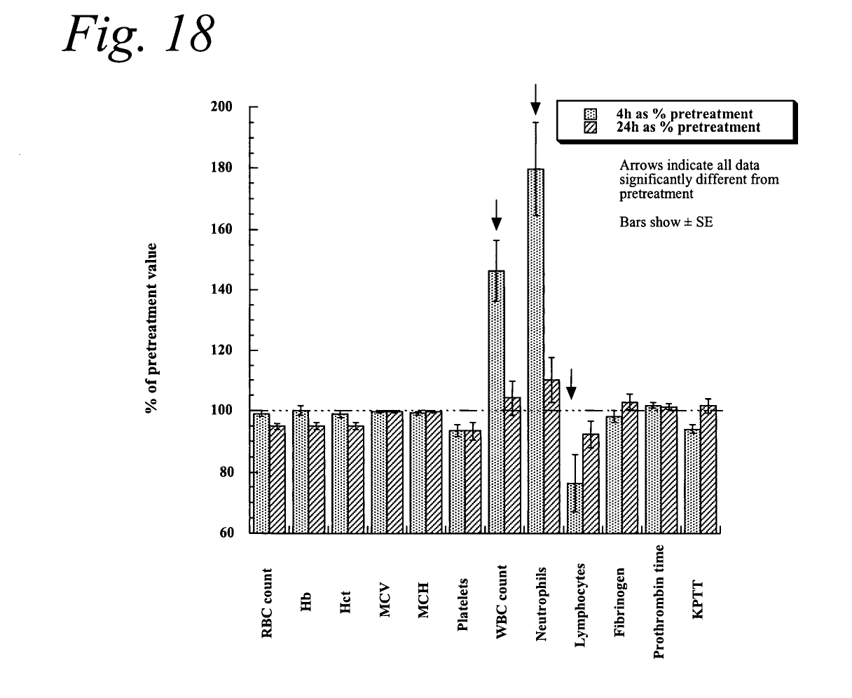

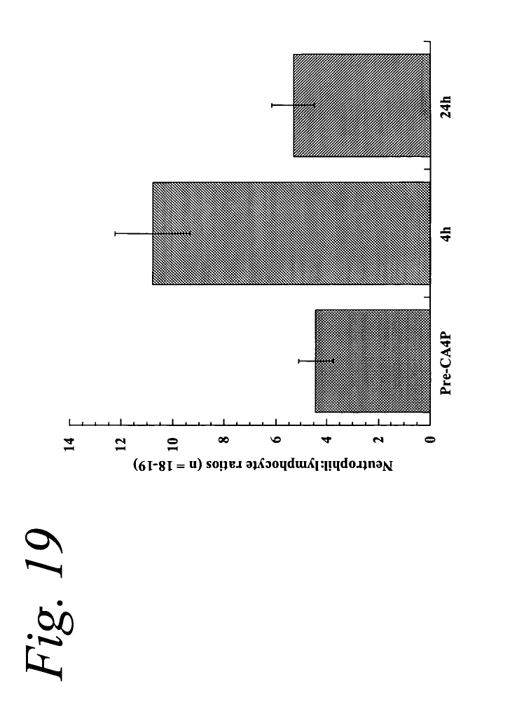

Cell counts were averaged from nineteen patients treated with a range of CA4P doses (52 to 114 mg/m²). The results of the complete hematological analysis obtained at 4 and 24 hours post-treatment are presented in FIG. 18 as a percentage of the corresponding pretreatment value. As indicated by the downward arrows, patients exhibited a highly significant increase in total white blood cell count at 4 hours following treatment with CA4P. The increase in white blood cells occurred despite a significant decrease in lymphocyte count (i.e. T and B-lymphocytes). Closer inspection revealed that the increase in total WBC count was due to a major increase in neutrophil count following treatment with CA4P. When expressed as a ratio, the data indicate that ratio of neutrophils to lymphocytes increase from approximately 4:1 to approximately 11:1 (see FIG. 19). The more than 2-fold increase in neutrophils and more than 20% decrease in lymphocytes was transient however, as white blood cell counts returned to normal levels within 24 hours of treatment.

Since neutrophils have a capacity to kill tumor cells through a number of cytotoxic and cytolytic mechanisms, neutrophil counts may be utilized as a prognostic indicator or predictor of therapeutic efficacy following treatment with a combretastatin or any other anti-vascular agent. Evidence of increased numbers of neutrophils (i.e. “neutrophilia”), if observed in the biological sample of a patient treated with a combretastatin, may be used to select the patient for continued treatment with the combretastatin. If the patient fails to exhibit neutrophilia, the information will be useful in determining whether the patient should continue to receive treatment or whether treatment should be discontinued. The reliability of neutrophilia as a marker (i.e. “biomarker”) of therapeutic efficacy may be validated by correlating the observation of enhanced numbers of neutrophils in the blood of large group of patients, with therapeutic efficacy in that group of patients.

### Example 5

### A Phase I/II Trial of CA4P in Combination with Carboplatin and Paclitaxel Chemotherapy in Patients with Advanced Cancer and Advanced Ovarian Carcinoma

A Phase I/II trial of CA4P in combination with carboplatin and paclitaxel was performed to establish the optimal dosage of this triple combination and assess its efficacy in patients with ovarian/primary peritoneal cancer who have relapsed following first line treatment with a regime comprising a platinum coordination compound in the adjuvant or metastatic setting.

The Phase I study population was comprised of two (2) cohorts of ten (10) subjects each to equal (20) adults (male and female) aged 18 or older. Each patient met the study entry criteria having had histopathologically or cytologically confirmed malignant solid tumors that have failed standard therapy or for which no life prolonging treatment exists. In addition, subjects had adequate organ function and were absent any other major concomitant illness. No clinically significant cardiac abnormality or evidence of QTc prolongation was evident. Each subject had a life expectancy of greater than 12 weeks, adequate bone marrow function (Absolute granulocyte count>1500 cells/mm³ and Platelet count>100,000 cells/mm³), adequate hepatic function (total bilirubin<1.5 mg/dl; ALT and AST<2.5×upper limit of normal), and adequate renal function (Glomerular Filtration Rate (GFR) measured by EDTA clearance>35 ml/min).

A minimum 28-day interval must have passed from the time the subject had last received chemotherapy and/or immunotherapy or a 14-day interval for radiotherapy prior to the first dose of study drugs (42 days for therapy known to be associated with delayed toxicity such as nitrosureas or mitomycin-C). Patients were excluded from the study if they have had a serious intercurrent infection(s) or other nonmalignant medical illness that is uncontrolled or whose control could be jeopardized by the complications of this therapy. Patients were excluded if they presented with Grade 2 or greater pre-existing peripheral neuropathy (motor or sensory), uncontrolled brain metastasis defined by continued symptoms or requirement for corticosteroids, major surgery within four weeks prior to receiving the first cycle of treatment, symptomatic peripheral vascular diseases or cerebrovascular disease, or a psychiatric disorder or other condition that renders the subject incapable of complying with the requirements of the protocol.

For the initial phase I study, one of two treatment arms (CA4P+Carboplatin and CA4P+Paclitaxel) were administered to patients in a dose-escalation trial. Dose escalation of carboplatin, paclitaxel and CA4P was performed until a Maximum Tolerable Dose (MTD) was established. Cycle time was 21 days. On Day 1, two groups of 3 patients received either a 10 minute infusion of one of 2 starting dose levels of CA4P. Group 1 received a 36 mg/m² free acid (equivalent to 44 mg/m² disodium salt) dose of CA4P when combined with carboplatin and Group 2 received a 27 mg/m² free acid (equivalent to 30 mg/m² disodium salt) dose of CA4P when combined with paclitaxel. On Day 2, Group 1 patients received a 1 hour infusion of carboplatin and Group 2 patients received a 3 hour infusion of paclitaxel. The starting dose of carboplatin was AUC 4 and the starting dose of paclitaxel will be 135 mg/m².

The total dose of carboplatin actually administered will correspond to a target area under the concentration-curve (AUC) and will be calculated using a modified Calvert formula: (target AUC)×(CrCl+25)=carboplatin dose per cycle in milligrams. Creatinine clearance (CrCL) is capped at 100 cc/minute and will be calculated using the Cockroft-Gault formula: (140−age×body mass)/(plasma creatinine×72)×GF. This formula provides the total dose carboplatin in milligrams (not mg/m2 dose).

Dose escalation was performed as outlined in Table III. If a dose-limiting toxicity (DLT) was seen in one patient, the cohort was to be expanded to six patients. In the absence of a DLT in one patient, a minimum of three patients were to be treated at each dose level. Subsequent dose levels were not to be opened until three patients at the

TABLE III
Phase I Dose Escalation Schedules
CA4P + Carboplatin Treatment Group

| | CA4P | Carboplatin |
|---|---|---|
| Dose Level | (free acid dose in mg/m²) | (dose in AUC) |
| 1 | 36 | 4 |
| 2 | 45 | 4 |
| 3 | 45 | 5 |
| 4 | 60 | 5 |
| 5 | 70 | 5 |

CA4P + Paclitaxel Treatment Group

| | CA4P | Paclitaxel |
|---|---|---|
| | (free acid dose in mg/m²) | (dose in mg/m²) |
| 1 | 27 | 135 |
| 2 | 27 | 175 |
| 3 | 36 | 175 |
| 4 | 45 | 175 |
| 5 | 60 | 175 |
| 6 | 70 | 175 |

CA4P/Carboplatin/Paclitaxel Treatment Group

| | CA4P | | |
|---|---|---|---|
| | (free acid dose in | | Paclitaxel |
| | mg/m²) | Carboplatin | (dose in mg/m² |
| 1 | 60 | 5 | 175 |
| 2 | 70 | 5 | 175 |
| (6 subjects)* | | | |

current dose level have completed administration of course 2. The maximum tolerable dose (MTD) was to be defined as the highest dose at which one or fewer patients experience a DLT. Once the MTD had been defined at or above 60 mg/m² CA4P in 3 patients that have been co-administered carboplatin and in three patients that have been co-administered paclitaxel, the dose of 60 mg/m² CA4P was to be assessed in combination with both paclitaxel (3 hour infusion) followed by carboplatin (1 hour infusion) in 3 patients.

For the phase II study, patients with Ovarian, Primary Peritoneal or Fallopian Tube Cancer who have relapsed following first line treatment with a regime comprising a platinum coordination compound in the adjuvant or metastatic setting, will be enrolled and administered a double (CA4P+Carboplatin or CA4P+Paclitaxel) or triple combination (CA4P+Carboplatin+Paclitaxel) of study drugs. The actual doses for the phase II trial will only be decided once the 60 mg/m² and 70 mg/m² cohorts are completed. If the MTD of CA4P is less than 60 mg/m² in either doublet, the lowest MTD will be used in the triplet. All three drugs will be combined at a Recommended Phase II dose (RP2D) and examined in a cohort of 6 patients. If no more than one DLT is seen, this dose will be taken forward into an additional 24 patients for the Phase II element of the study. If more than one DLT is seen with the triple drug combination in the first cohort of 6 patients at the RP2D, a reduction of dose level of one or more drugs will assessed in a further cohort of 6 patients before expansion into the Phase II element of the study. In any case, the maximum doses used for the triple combination in the phase II element of the study will be carboplatin AUC 5, paclitaxel 175 mg/m² and CA4P 70 mg/m².

CA4P will be randomly administered by infusion at its RP2D on Days 1, 8 and 15. On Day 2 patients will have a 60-minute infusion of carboplatin or a 3-hour infusion of paclitaxel or, if receiving both chemotherapeutic agents, a 3-hour infusion of paclitaxel followed by a 60-minute infusion of carboplatin. A treatment cycle will be 21 days and a maximum of 6 cycles of treatment will be administered. Any medications known to prolong QTc are to be withheld 72 hours prior to the intravenous administration of CA4P and will be resumed no earlier than 24 hours after dosing with CA4P. All patients must have adequate organ function and be absent any other major concomitant illness. Patient must not present with any clinically significant cardiac abnormality or evidence of QTc prolongation.

### Example 6

### A Phase II Trial of CA4P in Combination with Carboplatin and Paclitaxel Chemotherapy in Patients with Advanced Imageable Malignancies

Patients who have relapsed following first line treatment with a regime comprising a platinum coordination compound in the adjuvant or metastatic setting are enrolled in and administered one of two doses of CA4P in combination with Carboplatin and Paclitaxel.

CA4P will be randomly administered by infusion for 10 minutes at a free acid dose of 45 or 63 mg/m² on Days 1, 8, and 15. All dosages are calculated based on the mg of the free acid (non-solvated) form of CA4P. The total amount of drug administered in determined by multiplying the dose with the measured Body Surface Area (BSA) of the subject. BSA (i.e. m²) is determined using the Mosteller formula: BSA=([Height (cm)×Weight (kg)]/3600)^{1/2}. On Day 2 (21-28 hours post CA4P treatment), patients will have a 3-hour infusion of Paclitaxel (200 mg/m²) followed by a 60-minute infusion of Carboplatin (AUC=6). The dose of carboplatin will correspond to a target area under the concentration curve (AUC) and will be calculated using a modified Calvert formula: (target AUC)×(CrCl+25)=carboplatin dose per cycle in milligrams. Creatinine clearance (CrCL) is capped at 100 cc/minute and will be calculated using the Cockroft-Gault formula: (140−age×body mass)/plam creatinine×72)×GF, where GF is a gender correction factor. This formula provides the total dose of carboplatin in milligrams.

A treatment cycle will be 21 days and a maximum of 6 cycles of treatment are administered. Any medications known to prolong QTc are to be withheld 72 hours prior to the intravenous administration of CA4P and will be resumed no earlier than 24 hours after dosing with CA4P.

The tumor assessment schedule will be once every two weeks for two cycles (i.e. six weeks). Anti-tumor activity will be evaluated by a variety of methods, including tumor size, tumor perfusion, and the presence of validated biomarkers.

i) Assay Methods for Effects on Tumor Response

All subjects with measurable disease who have received a minimum of two cycles of treatment will be evaluated for tumor response, i.e. a change in tumor size. The effect of anti-tumor therapy on tumor size and anatomy can be determined by clinical examination and, preferably, clinical imaging. Clinical measurements should be taken using a rule or calipers and recorded in metric notation. Clinical tumor lesions will only be considered measurable when they are superficial (e.g. skin nodules, palpable lymph nodes). For the case of skin lesions, documentation by color photography including a rule to estimate the size of the lesion is recommended.

As used herein, “measurable disease” shall refer to the presence of at least one measurable lesion. A “measurable lesion” is defined as a lesion that can be accurately measured in at least one dimension with the longest diameter greater or equal to 20 mm and which is not classified as a bone lesion, a leptomeningeal disease, ascites, a pleural or pericardial effusion, an inflammatory breast disease, a lymphangitis cutis/pulmonis, an abdominal mass that is not confirmed and followed by imaging techniques, or a cystic lesion or lesion occurring within a previously irradiated area unless it is documented as a new lesion since the completion of radiation therapy.

All measurable lesions up to a maximum of five lesions per organ and ten lesions in total representative of all involved organs should be identified as target lesions to be measured and recorded at baseline. Target lesions should be selected based on their size (lesions with the longest diameter) and their suitability for accurate repeat assessment. At baseline, a sum of the longest diameters (LD) for all target lesions will be calculated and considered the baseline sum LD. The baseline sum LD will be used as the reference point to determine the objective tumor response of the measurable disease. Measurable lesions in excess of 10, and all sites of non-measurable disease, will be identified as non-target lesions. Non-target lesions will be recorded as “present” at baseline and should be evaluated at the same assessment time points as target lesions. The same method of assessment and the same technique will be used to identify and report each lesion at baseline and at re-assessment during treatment.

Conventional computerized tomography (CT) and/or magnetic resonance imaging (MRI) will be used to image measurable lesions using imaging cuts of 10 mm or less in slice thickness contiguously. Spiral CT will be performed using a 5 mm contiguous reconstruction algorithm. With a spiral CT scan, a lesion must be 10 mm in at least one dimension. Lesions on chest X-ray are acceptable as measurable lesions when they are clearly defined and surrounded by aerated lung. The anatomic imaging protocol will consist of a turbo spin echo breath-hold localizer sequence (5000 ms TR, 100 ms TE, 7 mm coronal slices, ˜3 mm in-plane resolution) for identification of the tumor region and determination of optimal field of view. A T1-weighted fat-saturated breath-hold FLASH sequence (100-250 ms TR) will be adjusted to achieve complete coverage of the tumor region over the breath-hold (2.3 ms out-of-phase and 4.6 ms in-phase TE, 7 mm axial slices, ˜1 mm in-plane resolution, 90 degree flip angle). A T2-weighted fat-saturated turbo spin echo sequence with respiratory gating (4000-6000 ms TR determined by respiration rate, 100 ms TE, 7 mm axial slices, ˜1 mm in-plane resolution) will be employed.

A tumor will be considered to have exhibited a Complete Response (CR) if all clinical and radiological evidence of target lesions has disappeared. Normalization of tumor marker level, if applicable, is also required. A tumor will have been considered to exhibit a Partial Response (PR) if the sum of the LD of all target lesions is decreased by 30% or greater in reference to the baseline sum LD. A tumor will be considered to exhibit Stable Disease (SD) if the tumor exhibits neither sufficient shrinkage to qualify for PR nor sufficient increase to qualify for Progressive Disease (PD). A tumor will be considered to exhibit Progressive Disease (PD) if the sum of LD of target lesions is increased at least 20% relative to the smallest sum of the LD recorded since treatment started or the appearance of one or more new lesions.

ii) Assay Methods for Effects on Tumor Perfusion

A series of time-resolved dynamic contrast enhanced images will be obtained using Dynamic Contrast Enhanced-Magnetic Resonance Imaging (DCE-MRI) to provide pharmacodynamic information on the distribution of a Gd-DTPA contrast agent within normal parenchyma and tumor tissue. The primary objective of this imaging protocol is to provide a measure of the response of tumor perfusion to therapy and to facilitate both quantitation of perfusion and correlation of tumor response with other therapeutic sequelae, ultimately including long-term response.

DCE-MRI will be performed on all tumors that are larger than 1 cm and that are unaffected by motion artifacts due to respiration, peristalsis or pulsatile flow (i.e. lungs and bowels are unacceptable). An initial screening DCE-MRI will be performed for baseline perfusion reading. Within 4 hours of receiving the first dose of CA4P, a DCE-MRI scan will be completed to document tumor perfusion. A second DCE-MRI scan will be completed just prior to cycle 3. An optional final follow-up scan may also be performed.

Tumor perfusion will be determined using a fast T1-weighted TurboFLASH sequence (4 ms TR, 1.5 ms TE, 7 mm slices, ˜1 mm in-plane resolution, 15 degree flip angle) in conjunction with injection of exogenous Gd-DTPA contrast agent. Sequential image acquisition will begin five seconds prior to bolus injection of 0.2 mmol/kg of contrast, and will proceed for 60 seconds at a frequency of 0.5/s, followed by 120 seconds at a frequency of 0.25/s, and finish with 180 seconds at 0.2/s for a total of 96 slices. Slice orientation will be chosen to optimize the visibility of tumor, surrounding parenchyma, and structures suitable for measurement of the tissue input function (e.g. hepatic artery/portal vein, spleen, inferior vena cava, abdominal aorta). Preliminary assessment of perfusion changes in response to therapy will compute relative perfusion in the tumor region of interest, normalized to input function. More quantitative analysis will involve kinetic compartment modeling of exchange and washout of contrast in tissue based on a modified Kety model.

iii) Biomarker Methods for Determining Anti-Tumor Activity

Blood samples are collected at several time points prior to CA4P infusion and at 4 hours post-CA4P infusion for each cycle. Whole blood is collected in tube containing an anticoagulant and a complete blood count including differential and platelet count is performed within one hour of collection. A complete lymphocyte count and a complete neutrophil count will be determined at 4 hours post-CA4P infusion.

### Conclusion

The above-described results readily demonstrate a variety of benefits, which may be achieved by combining one or more anticancer agents with a combretastatin compound. The anticancer agents can be effectively used to modulate tumor growth or metastasis of tumors that previously have developed a resistance to such drugs. Additionally, the present inventors have developed methods for the treatment of cancer, which permit the clinician to administer lowered dosages of anticancer agents with appropriate administration schedules thereby reducing unwanted side effects while maintaining efficacy.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. For example, other antivascular agents can be employed in the present invention in place of the combretastatin compounds.

1. A method for producing an anti-tumor effect in a patient suffering from cancer or tumor, the method comprising administering to the patient at least two anticancer agents and a combretastatin compound in amounts effective therefore.

2. The method as claimed in claim 1, wherein said at least two anti-cancer agents are selected from the group consisting of an alkylating agent, a bifunctional alkylating agent, a non-steroidal aromatase inhibitor, an immunotherapeutic agent, an antiangiogenic agent, a nitrosurea compound, an antimetabolite, an antitumor antibiotic, a mitotic inhibitor, radiation, a topoisomerase I inhibitor, and an anti-estrogen.

3. The method of claim 1, wherein said at least two anticancer agents comprises a taxane.

4. The method of claim 3, wherein said taxane is paclitaxel.

5. The method of claim 4, wherein paclitaxel is administered at a dose ranging from 135 mg/kg to 175 mg/kg.

6. The method of claim 5, wherein said at least two anticancer agents comprises a platinum coordination compound.

7. The method of claim 6, wherein platinum coordination compound is carboplatin.

8. The method of claim 7, wherein carboplatin is administered at a dose ranging from AUC 4 to AUC 6.

9. The method as claim 1, wherein said at least two anticancer agents comprise a taxane and a platinum coordination compound.

10. The method of claim 9, wherein said taxane is paclitaxel and said platinum coordination compound is carboplatin.

11. The method of claim 1, wherein said combretastatin compound is selected from the group consisting of CA1, CA4, CA1P, CA4P, or a prodrug or salt thereof.

12. The method of claim 1 wherein said combretastatin compound is administered at a dose ranging from between 45 mg/kg and 63 mg/kg.

13. The method of claim 1, wherein the at least two anticancer agents and the combretastatin compound are simultaneously or sequentially administered.

14. The method of claim 1, wherein said cancer is selected from the group consisting of ovarian cancer, fallopian tube cancer, cervical cancer, breast cancer, lung cancer, or primary cancer of the peritoneum.

15. A method for producing an anti-tumor effect in a patient with a tumor that is refractive to treatment with one or more anticancer agents, the method comprising administering to the patient the one or more anticancer agents together with a combretastatin compound in amounts effective therefore.

16. The method of claim 15, wherein said at least one anti-cancer agent is selected from the group consisting of an alkylating agent, a bifunctional alkylating agent, a non-steroidal aromatase inhibitor, an immunotherapeutic agent, an antiangiogenic agent, a nitrosurea compound, an antimetabolite, an antitumor antibiotic, a mitotic inhibitor, radiation, a topoisomerase I inhibitors, and an anti-estrogen.

17. The method of claim 15, wherein said at least one anticancer agent is a taxane.

18. The method of claim 17, wherein said taxane is paclitaxel.

19. The method of claim 18, wherein paclitaxel is administered at a dose ranging from 135 mg/kg to 175 mg/kg.

20. The method of claim 19, wherein said at least one anticancer agent is a platinum coordination compound.

21. The method of claim 20, wherein said platinum coordination compound is carboplatin.

22. The method of claim 21, wherein said carboplatin is administered at a dose ranging from AUC 4 to AUC 6.

23. The method as claim 15, wherein said at least one anticancer agent comprises both a taxane and a platinum coordination compound.

24. The method of claim 23, wherein said taxane is paclitaxel and said platinum coordination compound is carboplatin.

25. The method of claim 15, wherein said combretastatin compound is selected from the group consisting of CA1, CA4, CA1P, CA4P, or a prodrug or salt thereof.

26. The method of claim 15 wherein said combretastatin compound is administered at a dose ranging from between 45 mg/kg and 63 mg/kg.

27. The method of claim 15, wherein the at least one anticancer agent and the combretastatin compound are simultaneously or sequentially administered.

28. The method of claim 15, wherein said tumor is resistant to combretastatin.

29. The method of claim 15, wherein said tumor is resistant to a taxane.

30. The method of claim 15, wherein said tumor is resistant to a platinum coordination compound.

31. The method of claim 15, wherein said tumor is resistant to both a taxane and a platinum coordination compound.

32. The method of claim 15, wherein said tumor is a solid tumor selected from the group consisting of a melanoma, an ovarian tumor, a cervical tumor, a breast tumor, small cell lung tumor, a non-small cell lung tumor, a fallopian tube tumor, and a primary tumor of the peritoneum.

33. A method for producing an anti-tumor effect in an animal suffering from cancer, comprising administration of a combretastatin compound and at least two anticancer agents, in amounts effective therefore, wherein said combretastatin compound is administered at a time relative to administration of said at least two anticancer agents is sufficient to modulate blood flow to said tumor to provide a time-dependent effective tumor concentration of said anticancer agent.

34. The method of claim 33, wherein one of said at least two anticancer agents is a peak tumor concentration agent.

35. The method of claim 34, wherein said peak tumor concentration agent is administered simultaneously or in close temporal proximity to said combretastatin compound.

36. The method of claim 35, wherein said peak tumor concentration agent is a platinum coordination compound selected from the group consisting of cisplatin, oxaliplatin, and carboplatin.

37. The method of claim 33, wherein one of said at least two anticancer agents is a duration exposure agent.

38. The method of claim 37, wherein said duration exposure agent is administered after the administration of the combretastatin compound.

39. The method of claim 38, wherein said duration exposure agent is a taxane selected from the group consisting of paclitaxel and docetaxel.

40. The method of claim 33, wherein two of said at least two anticancer agents are duration exposure agent and a peak tumor concentration agent.

41. The method of claim 40, wherein said duration exposure agent is a platinum coordination compound selected from the group consisting of carboplatin, cisplatin, and oxaliplatin, and said peak tumor concentration agent is a taxane selected from the group consisting of paclitaxel and docetaxel.

42. The method of claim 40, wherein said duration exposure agent and said peak tumor concentration agent are administered after the administration of the combretastatin compound.

43. The method of claim 40, wherein said duration exposure agent and said peak tumor concentration agent are administered within 24 hours after the administration of the combretastatin compound.

44. The method of claim 33, wherein said combretastatin compound is selected from the group consisting of CA1, CA4, CA1P, CA4P, or a prodrug or salt thereof.

45. A pharmaceutical composition for producing an anti-tumor effect in an animal suffering from cancer, comprising at least two anticancer agents and a combretastatin compound, in amounts effective therefore in a pharmaceutically acceptable carrier.

46. The pharmaceutical composition as claimed in claim 45, wherein said at least two anticancer agents are selected from the group consisting of an alkylating agents, a bifunctional alkylating agents, a non-steroidal aromatase inhibitors, an immunotherapeutic agent, an antiangiogenic agent, a nitrosurea compound, an antimetabolites, an antitumor antibiotic, a mitotic inhibitor, radiation, a topoisomerase I inhibitors, and an anti-estrogen.

47. The pharmaceutical composition of claim 45, wherein said at least two anticancer agents comprise a platinum coordination compound and a taxane.

48. The pharmaceutical composition of claim 47, wherein said platinum coordination compound is carboplatin and said taxane is paclitaxel.

49. The pharmaceutical composition of claim 48, wherein paclitaxel comprises a unit dosage form of between 135 and 175 mg/kg.

50. The pharmaceutical composition of claim 49, wherein carboplatin comprises a unit dosage form between AUC 4 and AUC 6.

51. The pharmaceutical composition of claim 45, wherein said combretastatin compound is selected from the group consisting of CA1, CA4, CA1P, CA4P, or a prodrug or salt thereof.

52. The pharmaceutical composition of claim 51, wherein said combretastatin compound comprises a dosage unit form of between 45 and 63 mg/kg.

53. A method for determining the prognosis of a patient suffering from cancer, wherein said patient has been administered an anticancer agent, the method comprising:
(a) obtaining a biological sample from the patient;
(b) determining a granulocyte level of the biological sample;
(c) comparing the granulocyte level with a baseline level;
(d) correlating the granulocyte level with an indication of unfavorable prognosis if the granulocyte level is greater than the baseline level or correlating the neutrophil level with an indication of favorable prognosis if the granulocyte level is equal to or less than the baseline,
thereby determining the prognosis of the patient.

54. The method of claim 53, wherein said anti-cancer agent is a combretastatin.

55. The method of claim 53, wherein said granulocyte level is a neutrophil level.

56. The method of claim 53 wherein said biological sample is obtained less than 24 hours after treatment with the anti-cancer agent.

57. The method of claim 53 wherein said biological sample is obtained less than 6 hours after treatment with the anti-cancer agent.

58. A method for selecting a patient for further treatment with an anti-cancer agent, the method comprising:
(a) determining a granulocyte level in a first biological sample from the patient;
(b) administering the anti-cancer agent to the patient;
(c) determining a second granulocyte level from a second biological sample obtained from the patient;
(d) comparing the first and second granulocyte levels; and
(e) selecting the patient for further treatment if an increase in granulocyte level is observed.

59. The method of claim 58, wherein said anti-cancer agent is a combretastatin.

60. The method of claim 58, wherein said granulocyte level is a neutrophil level.

61. The method of claim 58, wherein said biological sample is obtained less than 24 hours after treatment with the anti-cancer agent.

62. The method of claim 58, wherein said biological sample is obtained less than 6 hours after treatment with the anti-cancer agent.

63. A method for monitoring the progression of a tumor in patient, the method comprising:
(a) determining a granulocyte level in a first biological sample from the patient;
(b) administering the anti-cancer agent to the patient;
(c) determining a second granulocyte level from a second biological sample obtained from the patient; and
(d) comparing the first and second granulocyte levels,
thereby monitoring the progression of the tumor in the patient.

64. The method of claim 63, wherein said anti-cancer agent is a combretastatin.

65. The method of claim 63, wherein said granulocyte level is a neutrophil level.

66. The method of claim 63, wherein said biological sample is obtained less than 24 hours after treatment with the anti-cancer agent.

67. The method of claim 63, wherein said biological sample is obtained less than 6 hours after treatment with the anti-cancer agent.
